(19) United States
(12) Patent Application Publication
(10) Pub. No.: US 2025/0215479 A1
(43) Pub. Date: Jul. 3, 2025

(54) Methods for Single-Molecule Amplification of Nuclear RNA

(71) Applicant: Northeastern University, Boston, MA (US)

(72) Inventors: Yifang Liu, West Roxbury, MA (US); Sara H. Rouhanifard, Chestnut Hill, MA (US)

(21) Appl. No.: 19/003,520

(22) Filed: Dec. 27, 2024

Related U.S. Application Data
(60) Provisional application No. 63/616,986, filed on Jan. 2, 2024.

(51) Int. Cl. C12Q 1/6818; C12Q 1/6806; C12Q 1/6876

(52) U.S. Cl. CPC C12Q 1/6818; C12Q 1/6806; C12Q 1/6876

(57) ABSTRACT

Provided herein are methods of labeling nuclear RNA, including methods comprising: (a) isolating a nucleus of a cell, the nucleus comprising nuclear RNA; (b) contacting the nucleus with a fixative, thereby producing a fixed nucleus; (c) contacting the fixed nucleus with a surfactant; and (d) hybridizing a primary probe to the nuclear RNA, thereby forming a primary sample comprising labeled nuclear RNA.

### RELATED APPLICATION

This application claims the benefit of U.S. Provisional Application No. 63/616,986, filed on Jan. 2, 2024, which is incorporated by reference herein in its entirety.

### INCORPORATION BY REFERENCE OF MATERIAL IN XML

This application incorporates by reference the Sequence Listing contained in the following extensible Markup Language (XML) file being submitted concurrently herewith:
a) File name: 52002394-001.xml; created Dec. 27, 2024, 207,946 Bytes in size.

### BACKGROUND

Single-molecule fluorescent in situ hybridization (FISH) is the gold standard for imaging and quantifying RNA molecules inside cells. However, the total fluorescence produced is insufficient for high-throughput methods. For example, separation of cells based on RNA expression using flow cytometry or high throughput imaging using low magnification microscopy. There is a need for improved FISH-based amplification strategies that maintain specificity but greatly enhance fluorescent signal, with improved ability to access and amplify nuclear RNAs.

### SUMMARY

Transcriptional bursts refer to periods when RNA polymerase interacts with a DNA locus, leading to active gene transcription. This bursting activity can vary across individual cells, and analyzing the differences in transcription sites can help identify key drivers of gene expression for a specific target. Scaffolding methods based on fluorescence in situ hybridization (FISH) have been widely used to amplify the fluorescent signal of mRNAs and sort cells based on mRNA expression levels. However, these methods are inefficient at targeting nuclear RNA, including transcription sites, due to the probes' limited accessibility through cellular compartment membranes and crosslinked proteins. Additionally, the required formaldehyde fixation interferes with downstream analysis of chromatin and protein-binding interactions. To address these challenges, a platform that integrates amplified FISH with reversible crosslinkers and allows access to the nucleus is needed. In some embodiments, nuclear clampFISH (nuclampFISH) method disclosed herein amplifies the fluorescent signal of mRNAs using a reversible crosslinker, enabling the sorting of cells based on nuclear RNA expression and compatible with downstream biochemical analysis. This assay demonstrates that transcriptionally active cells have more accessible chromatin for a respective gene. The methods disclosed herein are highly accessible and do not require specialized computation or equipment.

Disclosed herein are methods for amplifying the signal (e.g., fluorescent signal) of nuclear RNAs. In some embodiments, the signal of nuclear RNAs are amplified with high sensitivity while maintaining high specificity.

In some embodiments disclosed herein are methods of labeling nuclear RNA, the methods comprising: (a) isolating a nucleus of a cell, the nucleus comprising nuclear RNA; (b) contacting the nucleus with a fixative, thereby producing a fixed nucleus; (c) contacting the fixed nucleus with a surfactant; and (d) hybridizing a primary probe to the nuclear RNA, thereby forming a primary sample comprising labeled nuclear RNA.

In some embodiments, the primary probe is a primary click-amplified Fluorescence In Situ Hybridization (clampFISH) probe, a Hybridization Chain Reaction (HCR) probe, or a Systematic Amplification of Bioorthogonal Reporting (SABER) probe.

In some embodiments, methods of the present disclosure further comprise: (e) contacting the primary sample with an agent that locks the primary probe to the nuclear RNA.

In some embodiments, methods of the present disclosure further comprise: (f) contacting the primary sample with (i) a set of secondary probes or (ii) a set of secondary probes and an agent that locks the set of secondary probes to the primary probe, thereby forming a secondary sample.

In some embodiments, the secondary probe is a secondary clampFISH probe, a HCR probe, or a SABER probe.

In some embodiments, methods of the present disclosure further comprise: (g) contacting the secondary sample with (i) a set of tertiary probes or (ii) a set of tertiary probes and an agent that locks the set of tertiary probes to each secondary probe, thereby forming a tertiary sample; (h) contacting the tertiary sample with (i) a set of secondary probes or (ii) a set of secondary probes and an agent that locks the set of secondary probes to each tertiary probe; and (i) repeating steps (g) and (h) until a desired level of fluorescent signal is achieved.

In some embodiments, the tertiary probe is a tertiary clampFISH probe, a HCR probe, or a SABER probe.

In other embodiments disclosed herein are methods of detecting nuclear RNA, the method comprising: (a) isolating a nucleus of a cell, the nucleus comprising a nuclear RNA; (b) contacting the nucleus with a fixative, thereby producing a fixed nucleus; (c) contacting the fixed nucleus with a surfactant; (d) hybridizing a primary probe to the nuclear RNA, thereby forming a primary sample comprising labeled nuclear RNA; and (e) detecting the labeled nuclear RNA.

In some embodiments, detecting labeled nuclear RNA comprises using flow cytometry, microscopy, or a combination thereof.

In yet other embodiments disclosed herein are methods of analyzing nuclear RNA, the method comprising: (a) isolating a nucleus of a cell, the nucleus comprising nuclear RNA; (b) contacting the nucleus with a fixative, thereby producing a fixed nucleus; (c) contacting the fixed nucleus with a surfactant; (d) hybridizing a primary probe to the nuclear RNA, thereby forming a primary sample comprising labeled nuclear RNA; (e) detecting the nuclear RNA; (f) contacting the fixed nucleus with a reducing agent, thereby unfixing the nucleus; and (g) analyzing the nuclear RNA.

In some embodiments, analyzing nuclear RNA comprises using mass spectrometry, a biochemical assay, chromatin profiling, RNA sequencing, metabolomics, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows an example scheme of transcriptional bursts, in one embodiment.

FIGS. 2A-C illustrate FISH-based methods to detect transcription sites: FIG. 2A: smFISH, FIG. 2B: clampFISH, and FIG. 2C: Hybridization chain reaction (HCR) FISH. FIG. 2D illustrates overlaid FISH spots in mRNA exon and intron, which indicate transcription (txn) sites. FIG. 2E shows images of EEF2 exons and smFISH images of EEF2 introns which show colocalization and transcription sites. FIG. 2F is an overlaid histogram plot of exon RNA count distribution for clampFISH, HCR FISH, and smFISH for two biological replicates.

FIG. 3A shows clampFISH images of EEF2 exon and smFISH images of EEF2 intron without Pla B treatment. The arrows indicate the transcription sites. FIG. 3B shows clampFISH images of EEF2 exon and smFISH images of EEF2 intron after Pla B treatment.

FIG. 3C shows NuclampFISH images of EEF2 exon after nucleus isolation and smFISH images of EEF2 intron. FIG. 3D shows NuclampFISH images of EEF2 exon after nucleus isolation and modified hybridization parameters, and smFISH images of EEF2 intron. FIG. 3E shows a workflow of a nuclampFISH method, in one example embodiment. FIG. 3F shows a timeline of nuclampFISH process, in one embodiment. FIG. 3G shows amplification efficiency of nuclampFISH, two rounds, four rounds, six rounds, eight rounds of nuclampFISH images are shown. FIG. 3H shows a box and whisker plot of nuclampFISH spot intensities in each round of amplification. n=3 biological replicates.

FIGS. 4A-B show that smFISH signal of EEF2 intron increases after Pla B treatment. FIG. 4A shows a histogram of single spot intensities of smFISH with Pla B and without Pla B treatment in FIGS. 3A and 3B. FIG. 4B shows a bar graph of mean intensities of smFISH spots with Pla B and without Pla B treatment. n=3 biological replicates (bars represent mean±SD).*p<0.05, **p<0.01, ***p<0.001, ****p<0.0001.

FIG. 5A-B show optimized conditions of nuclampFISH with Triton™ X-100 and increased SSC concentration. FIG. 5A shows improvement of colocalization ratio of nuclampFISH after nucleus isolation, adding triton and increasing SSC concentration. FIG. 5B shows a histogram of single spot intensities after treatment of triton and increased SSC concentration. n=3 biological replicates (bars represent mean±SD).

FIG. 6A shows NuclampFISH for long non-coding RNA NEAT1. a. ClampFISH images and nuclampFISH images of NEAT1. FIG. 6B shows flow cytometry data of nuclampFISH, dot plot of nuclampFISH signal compared to no primary negative control group, based on the low and high signal in the flow cytometry, sorted cells were imaged.

FIG. 7 shows the detection of CPSI intron using nuclampFISH method, in one example embodiment.

FIG. 8 shows NuclampFISH images for TMSF1 genes. NuclampFISH images of TMSF1 exon were overlapped with smFISH images of TMSF1 intron. Arrows indicate the transcription sites overlaid in both images.

FIG. 9 shows the compatibility of different crosslinkers for clampFISH. Images of different fixation reagents of clampFISH for HIST1H4E gene are shown.

FIGS. 10A-C show the application of a reversible crosslinker to nuclampFISH. FIG. 10A is a photomicrograph of HeLa cells stained for HIST1H4E mRNA using clampFISH after DSP (left) and formaldehyde crosslinking (right). FIG. 10B is a histogram of HIST1H4E mRNA counts per cell in DSP crosslinked HeLa cells versus formaldehyde crosslinked HeLa cells. FIG. 10C shows NuclampFISH images of EEF2 intron after nucleus isolation and smFISH images of EEF2 intron.

FIG. 11A is an example workflow of nuclampFISH, flow sorting, and chromatin accessibility assay. FIG. 11B is a flow cytometry plot (pseudocolored), Side Scatter (SSC) versus EEF2 intron nuclampFISH signal) showing a gating strategy for EEF2 intron mRNA in HeLa cells. Low, medium, and high expressers were gated as G1, G2, and G3, respectively, with fluorescent micrographs of G1, G2, and G3 cell populations after sorting. FIG. 11C shows a (left) bar graph of quantified nuclampFISH signal in each cell population after flow sorting and (right) correlation of RT-pPCR counts with nuclampFISH signal intensities after flow sorting. FIG. 11D shows the ratio of qPCR counts of nuclease treatment to no nuclease treatment to determine the quantity of chromatin open level and to quantify chromatin accessibility for the G1 and G3 groups. FIG. 11E shows a cartoon modeling scheme for chromatin openness for G1 and G3 groups. n=3 biological replicates (mean±SD).

FIG. 12 shows the correlation of RT-pPCR counts with nuclampFISH spots counts after flow sorting. n=3 biological replicates (mean±SD).

FIG. 13 shows a quantitative comparison of chromatin accessibility between DSP without reversal crosslinking and DSP with reversal crosslinking. **p<0.01. DSP reversal crosslinking is compatible with chromatin accessibility assay.

FIG. 14 shows an example of nuclampFISH being used to amplify nuclear RNA signals by overcoming barriers to nuclear access, enabling cell sorting based on transcriptional activity, and reversible crosslinking which enables chromatin analysis, according to an embodiment.

### DETAILED DESCRIPTION

A description of example embodiments follows.

Several aspects of the present disclosure are described below, with reference to examples for illustrative purposes only. It should be understood that numerous specific details, relationships, and methods are set forth to provide a full understanding of the present disclosure. One having ordinary skill in the relevant art, however, will readily recognize that the present disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure. Many of the techniques and procedures described, or referenced herein, are well understood and commonly employed using conventional methodology by those skilled in the art.

As described herein, in some embodiments, nuclampFISH, an amplified FISH method for targeting nuclear RNA and transcription sites has been established. This method achieves the goal of specific detection of nuclear RNA (including transcription sites) and amplifying the FISH signal of nuclear RNA (including transcription sites). Based on the specific and amplified nuclampFISH signal, a platform is offered herein to separate cells according to the expression level of transcription sites for downstream analysis, e.g., to understand transcription better. In some embodiments, FISH detection and chromatin analysis are combined with a chemical reversible crosslinker, e.g., DSP. In some embodiments, FISH detection and chromatin analysis are combined with a different crosslinker, e.g., DSSO. Other probes and amplification schemes (e.g., hybridization chain reaction (HCR)) may be compatible with methods of the present disclosure.

As demonstrated herein, when compared to clampFISH and HCR FISH for transcription sites, nuclampFISH can significantly increase the signal and achieve specific detection. Moreover, nuclampFISH can maintain the exponential amplification capacity of clampFISH. These methods can detect multiple nuclear RNAs, such as the lncRNA NEAT1. However, the total fluorescence produced for amplification strategies targeting NEAT1 is lower than expected based on the amplification increase for cytoplasmic RNAs, suggesting that the labeling efficiency is also lower for nuclear RNAs.

Next, the cells were sorted based on the expression level of transcriptional bursts. The accuracy of the separation was demonstrated by both RT-qPCR and imaging. After sorting and collecting nuclei based on expression levels, the crosslinking was reversed and a chromatin accessibility assay for the gene of interest was performed. Transcriptionally active cells had more open levels of chromatin than the transcription inactive cells. This provides direct evidence that open chromatin regions enable the binding of transcription factors and other regulatory elements to drive the transcription of a given gene. In the stochastic bursting model, a simplistic model fits kinetic information in a two-state system where the promoter alternates between an ON and OFF position [25]. This model has been expanded to include a third, ‘refractory’ promoter state where the promoter is bound but resistant to activation (i.e., no burst). The data described herein shows that the cells exhibiting no bursting have significantly less “open chromatin” than the bursting cells, with levels comparable to that of negative control, HPRT1. This supports the two-state model.

The example platform provided herein combines single-cell analysis with bulk-cell sensitivity to move beyond an RNA-centric view of transcription and include the interacting factors of chromatin and protein in the analysis.

In some embodiments, describe herein is a method for the specific detection of nuclear RNA and transcription sites. This assay broadens the application of clampFISH for transcription sites by amplifying specific FISH signals, thus enabling the separation of the cells for downstream analysis, including chromatin analysis, proteomics, and transcriptional profiling.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

When introducing elements disclosed herein, the articles “a,” “an,” “the,” and “said” are intended to mean that there are one or more of the elements. Further, the one or more elements may be the same or different. For example, unless the context clearly indicates otherwise, “a polypeptide” includes a single polypeptide, and two or more polypeptides.

Throughout this specification and the claims which follow, unless the context requires otherwise, the term “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, the term “consisting of” excludes any element, step, or ingredient not specified in the claim element. When used herein, the term “consisting essentially of” does not exclude materials or steps that do not materially affect the basic and novel characteristics of the claim.

Also provided herein are corresponding embodiments for each and every embodiment featuring the term “comprising,” “containing,” “including,” or “having,” wherein those terms are replaced by the term “consisting of” and/or “consisting essentially of”.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” “fewer than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the term “about” means within an acceptable error range for a particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of +20%, e.g., +10%, +5% or +1% of a given value. It is to be understood that the term “about” can precede any particular value specified herein, except for particular values used in the Exemplification. When “about” precedes a range, as in “90-99.9%,” the term “about” should be read as applying to both given values of the range, such that “about 90-99.9%” means about 90% repeats to about 99.9%.

As used herein, “a set” includes one or more objects (e.g., one probe, a plurality of probes, etc.) and one or more types of objects (e.g., a primary clampFISH probe, a primary clampFISH probe and a primary HCR probe).

As used herein, the term “locks” refers to an interaction (e.g., covalent bond) formed between two regions of a probe(s) around one or more regions of nuclear RNA or one or more other probes. For example, a first clampFISH probe (e.g., primary, secondary, tertiary clampFISH probe) may be locked to one or more other clampFISH probes or nuclear RNA via the formation of a covalent bond between the 3′ and 5′ ends (e.g., 3′ azide end and 5′ alkyne end) of the first clampFISH probe. In some embodiments, an agent (e.g., a click chemistry agent) connects (e.g., covalently binds) the end groups (e.g., azide and alkyne groups) located at the 3′ and 5′ ends of the first clampFISH probe. In some embodiments, a ring (e.g., 1,2,3-triazole ring) is formed between the end groups.

The term “subject” refers to a mammalian subject, preferably human.

Compounds described herein include those described generally, and are further illustrated by the classes, subclasses, and species disclosed herein. As used herein, the following definitions shall apply unless otherwise indicated. For purposes of the present disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

As used herein, the term “sample” refers to any sample that can be from or derived from a subject (e.g., a mammalian subject such as a human subject). The methods disclosed herein can be performed using a variety of possible sample types. For example, a bodily fluid, a single cell or cell lysate, a population of cells, a cell culture, and/or a tissue.

The phrase “pharmaceutically acceptable” means that the substance or composition the phrase modifies is, within the scope of sound medical judgment, suitable for use in contact with the tissues of humans and lower animals without undue toxicity, irritation, allergic response, and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the agents/compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, a “polynucleotide” is defined as a plurality of nucleotides and/or nucleotide analogs linked together in a single molecule. In some embodiments, a polynucleotide disclosed herein comprises deoxyribonucleotides. In some embodiments, the polynucleotide comprises ribonucleotides. Non-limiting examples of polynucleotides include single-, double- or multi-stranded DNA or RNA, DNA-RNA hybrids (e.g., each “T” position may be independently substituted by a “U” or vice versa), or a polymer comprising purine and pyrimidine bases, or other natural, chemically or biochemically modified, non-natural, or derivatized nucleotide bases. The backbone of the polynucleotide can comprise sugars and phosphate groups, modified or substituted sugar or phosphate groups, a polymer of synthetic subunits such as phosphoramidates, or a combination thereof.

As used herein, the term “nucleotide analog” or “altered nucleotide” or “modified nucleotide” refers to a non-standard nucleotide, including non-naturally occurring ribonucleotides or deoxyribonucleotides. A nucleotide analog may be modified at any position so as to alter certain chemical properties of the nucleotide yet retain the ability to perform its intended function. Non-limiting examples of positions of the nucleotide which may be derivatized include the 5 position, e.g., 5-(2-amino) propyl uridine, 5-bromo uridine, 5-propyne uridine, and 5-propenyl uridine; the 6 position, e.g., 6-(2-amino) propyl uridine; the 8-position for adenosine and/or guanosines, e.g., 8-bromo guanosine, 8-chloro guanosine, and 8-fluoroguanosine. Nucleotide analogs also include deaza nucleotides, e.g., 7-deaza-adenosine; O- and N-modified (e.g., alkylated or N6-methyl adenosine) nucleotides.

As used herein, the term “complementary” refers to sequence complementarity between two different polynucleotides or between two regions of the same polynucleotide. A first region of a polynucleotide is complementary to a second region of the same or a different polynucleotide if, when the two regions are arranged in an anti-parallel fashion, at least one nucleotide residue of the first region is capable of base pairing (i.e., hydrogen bonding) with a residue of the second region, thus forming a hydrogen-bonded duplex.

As used herein, the term “sequence identity” refers to the extent to which two nucleotide sequences have the same residues at the same positions when the sequences are aligned to achieve a maximal level of identity, expressed as a percentage. For sequence alignment and comparison, typically one sequence is designated as a reference sequence, to which test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

As used herein, the term “polypeptide” refers to a polymer of at least two amino acids covalently linked by an amide bond, regardless of length or post-translational modification (e.g., glycosylation or phosphorylation). A polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl, and/or other functional groups on a polypeptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a polypeptide can also be derivatized (e.g., alkylated) or labeled (e.g., with a detectable label, such as a fluorogen or a hapten) using methods known in the art. A polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a polypeptide can be an analog of a known and/or naturally-occurring peptide, for example, a peptide analog having conservative amino acid residue substitution(s).

As used herein, the term “antibody mimetic” refers to polypeptides capable of mimicking an antibody's ability to bind an antigen, but structurally differ from native antibody structures. Examples of antibody mimetics include, but not limited to, Adnectins, Affibodies, Affilins, Affimers, Affitins, Alphabodies, Anticalins, Avimers, DARPins, Fynomers, Kunitz domain peptides, monobodies, nanobodies, nanoCLAMPs, and Versabodies.

A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. In some embodiments, a pharmaceutical composition suitable for use in methods disclosed herein further comprises one or more pharmaceutically acceptable carriers.

“Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., polynucleotide) is administered. Such vehicles may be liquids, such as water and oils, including those of petroleum, animal, vegetable or synthetic origin, such as peanut oil, soybean oil, mineral oil, sesame oil, and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers are acetic acid, citric acid, formic acid, succinic acid, phosphoric acid, carbonic acid, malic acid, aspartic acid, histidine, boric acid, Tris buffers, HEPPSO, and HEPES.

Non-limiting examples of antioxidants are ascorbic acid, methionine, cysteine hydrochloride, sodium bisulfate, sodium metabisulfite, sodium sulfite, lecithin, citric acid, ethylenediamine tetraacetic acid (EDTA), sorbitol, and tartaric acid.

Non-limiting examples of amino acids are histidine, isoleucine, methionine, glycine, arginine, lysine, L-leucine, tri-leucine, alanine, glutamic acid, L-threonine, and 2-phenylamine.

Non-limiting examples of surfactants are polysorbates (e.g., polysorbate-20 or polysorbate-80); polyoxamers (e.g., poloxamer 188); Triton™; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl, and the like), benzalkonium chloride, benzethonium chloride, sodium dehydroacetate, and thimerosal, or mixtures thereof.

Non-limiting examples of saccharides are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, or iso-maltulose.

Non-limiting examples of salts are acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like. In some embodiments, the salt is sodium chloride (NaCl).

Agents (e.g., polynucleotides) described herein may be prepared in accordance with standard procedures and are administered at dosages that are selected to reduce, prevent, or eliminate, or to slow or halt progression of, a condition being treated (see, e.g., Remington's Pharmaceutical Sciences, Mack Publishing Company, Easton, PA, and Goodman and Gilman's The Pharmaceutical Basis of Therapeutics, McGraw-Hill, New York, N.Y., the contents of which are incorporated herein by reference, for a general description of methods for administering various agents for human therapy).

“Administering” or “administration,” as used herein, refers to providing a compound, composition, or pharmaceutically acceptable salt thereof described herein to a subject in need of treatment or prevention. Administering can be performed, for example, once, a plurality of times, and/or over one or more extended periods. Administration includes both direct administration (including self-administration), and indirect administration (including an act of prescribing a drug or directing a subject to consume an agent). For example, as used herein, one (e.g., a physician) who instructs a subject (e.g., a human patient) to self-administer an agent (e.g., a drug), or to have an agent administered by another and/or who provides a patient with a prescription for a drug is administering an agent to a subject.

“A therapeutically effective amount,” “an effective amount” or “an effective dosage” refers to an amount effective, at dosages and for periods of time necessary, to achieve a desired therapeutic result (e.g., treatment, healing, inhibition or amelioration of physiological response or condition, etc.). A full therapeutic effect does not necessarily occur by administration of one dose and may occur only after administration of a series of doses. Thus, a therapeutically effective amount may be administered in one or more administrations. A therapeutically effective amount may vary according to factors such as the disease state, age, sex, and weight of a mammal (e.g., a human patient), mode of administration, and the ability of a therapeutic or a combination of therapeutics to elicit a desired response.

An effective amount of an agent to be administered can be determined by a clinician of ordinary skill using the guidance provided herein and other methods known in the art. Relevant factors include the given agent, the pharmaceutical formulation, the route of administration, the type of disease or disorder, the identity of the subject (e.g., age, sex, weight) or host being treated, and the like. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Desired response or desired results include effects at the cellular level, tissue level, or clinical results. As such, “a therapeutically effective amount” or synonym thereto depends upon the context in which it is being applied. For example, in some embodiments it is an amount of the composition sufficient to achieve a treatment response as compared to the response obtained without administration of the composition. In other embodiments, it is an amount that results in a beneficial or desired result in a subject as compared to a control. As defined herein, a therapeutically effective amount of a composition (e.g., a pharmaceutical composition) disclosed herein may be readily determined by one of ordinary skill by routine methods known in the art. Dosage regimen and route of administration may be adjusted to provide the optimum therapeutic response.

“Treating” or “treatment,” as used herein, refers to taking steps to deliver a therapy to a subject, such as a mammal, in need thereof (e.g., as by administering to a mammal one or more therapeutic agents). “Treating” or “treatment” includes inhibiting the disease or condition (e.g., as by slowing or stopping its progression or causing regression of the disease or condition) and relieving the symptoms resulting from the disease or condition.

The term “treating,” or “treatment” refers to the medical management of a subject with the intent to improve, ameliorate, stabilize (i.e., not worsen), prevent, or cure a disease, pathological condition, or disorder-such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

“Mass Spectrometry” (MS) is a technique for measuring and analyzing molecules to produce a mass spectrum that serves as a “molecular fingerprint.” MS involves fragmenting a target molecule, then analyzing the fragments, based on their mass/charge ratios. A “mass spectrum” is a plot of data produced by a mass spectrometer. In some embodiments, a mass spectrum contains m/z values on x-axis and intensity values on y-axis. The term “m/z” or “mass-to-charge” ratio refers to the dimensionless quantity formed by dividing the mass number of an ion by its charge number. In liquid chromatography-mass spectrometry-based (LC-MS) metabolomics, a group of ions may originate from the same compound, and one compound can be represented by multiple peaks in LC-MS data with distinct m/z values but, at similar retention times, due to the presence of adducts (e.g., H⁺, Na⁺ or K⁺). A “peak” is a point on a mass spectrum with a relatively high y-value.

The term “fluorophore” as used herein refers to a composition that is inherently fluorescent or demonstrates a change in fluorescence upon binding to a biological compound or metal ion, i.e., fluorogenic. Fluorophores may contain substituents that alter the solubility, spectral properties or physical properties of the fluorophore. Numerous fluorophores are known to those skilled in the art and include, but are not limited to coumarin, cyanine, benzofuran, a quinoline, a quinazolinone, an indole, a benzazole, a borapolyazaindacene and xanthenes including fluorescein, rhodamine and rhodol as well as other fluorophores known in the art.

A “portion” of a polynucleotide means at least at least about five to about fifty sequential nucleotide residues of the polynucleotide. It is understood that a portion of a polynucleotide may include every nucleotide residue of the polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label,” as used herein, refers to a chemical moiety or protein that is directly or indirectly detectable (e.g., due to its spectral properties, conformation or activity) when attached to a target or compound and used in the present methods, including reporter molecules and carrier molecules. The label can be directly detectable (e.g., fluorophore) or indirectly detectable (e.g., hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent labels (e.g., fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate such as Amplex Red reagent (Molecular Probes, Inc.) to detect the presence of HRP. Numerous labels are known by those of skill in the art and include, but are not limited to, particles, fluorophores, haptens, enzymes and their calorimetric, fluorogenic and chemiluminescent substrates and other labels known in the art.

Transcription sites are specific loci where the RNA polymerase binds to the DNA. The detection of a transcription site by methods such as RNA FISH indicates that these genes are transcriptionally active or “bursting.” It is well established in the field that single cells have high variability in the expression level of transcription sites, meaning they are in distinct cellular states [1-4]. However, the specific mechanisms and regulatory machinery behind such variation are less understood. Transcription regulation, specifically bursting, involves the recruitment and release of RNA Pol II at the promoter. When this activity is very high, the expression of bursts can appear homogeneous, but when they are less frequent, it leads to cell-to-cell variability [5]. One of the major challenges is to understand the regulators of bursting.

Current methods for studying transcription sites primarily use RNA Pol II chromatin immunoprecipitation coupled with sequencing (ChIP-seq) [6,7]. Other methods are based on nascent RNA enrichment and sequencing, which can track newly transcribed RNAs [8]. However, while these methods are genome-wide and provide details about DNA-protein interactions, these are bulk methods that lack single-cell resolution. Single-molecule fluorescence in situ hybridization (smFISH) is an imaging-based method for measuring the frequency and duration of transcription sites in single cells by targeting the introns of transcripts of interest [9,10]. It can provide information on spatial localization and absolute quantification of transcripts by detecting single molecules of RNA inside fixed cells. However, this method does not inform about RNA-protein interactions, and the smFISH signal is not bright enough to separate cells based on transcriptional activity using flow cytometry, which would enable biochemical assays. An ideal method for studying transcriptional bursts would be gene-specific, sensitive, and specific enough for flow cytometry, inform on single-cell activity, and have compatibility with biochemical assays for downstream analysis.

Various FISH-based scaffolding methods have been developed to enhance the fluorescent signals produced from RNA-binding oligonucleotide probes. These methods can all achieve high amplification [11-13] but lose signal integrity in nuclear RNA. One of these methods is click-amplified FISH (clampFISH), which uses a “C” shaped probe to hybridize and form a double helix with the target RNA, followed by ligation using bioorthogonal click chemistry [14,15]. The benefit of this method is that the ligated probes can survive stringent wash steps and achieve exponential amplification, and the backbone sequence can be modified for facile multiplexing. This method has also been recently expanded for faster analysis and higher throughput (clampFISH 2.0) [15]. Examples of clampFISH are disclosed in US. Pub. No. US 2019/0382838 A1, which is incorporated by reference herein in its entirety.

The present disclosure relates, in part, to methods of labeling nuclear RNA (e.g., nuclear RNA from a subject), the methods comprising:
(a) isolating a nucleus of a cell (e.g., a cell from a subject, a mammalian cell), the nucleus comprising nuclear RNA;
(b) contacting the nucleus with a fixative, thereby producing a fixed nucleus;
(c) contacting the fixed nucleus with a surfactant; and
(d) hybridizing a primary probe to the nuclear RNA, thereby forming a primary sample comprising labeled nuclear RNA.

In some embodiments, the primary probe is a primary click-amplified Fluorescence In Situ Hybridization (clampFISH) probe, a Hybridization Chain Reaction (HCR) probe, or a Systematic Amplification of Bioorthogonal Reporting (SABER) probe. In some embodiments, the primary probe is a primary clampFISH probe.

In some embodiments, methods of the present disclosure further comprise:
(e) contacting the primary sample with an agent that locks the primary probe to the nuclear RNA.

In some embodiments, methods of the present disclosure further comprise:
(f) contacting the primary sample with (i) a set of secondary probes or (ii) a set of secondary probes and an agent that locks the set of secondary probes to the primary probe, thereby forming a secondary sample.

In some embodiments, the secondary probe is a secondary click-amplified Fluorescence In Situ Hybridization (clampFISH) probe, a Hybridization Chain Reaction (HCR) probe, or a Systematic Amplification of Bioorthogonal Reporting (SABER) probe. In some embodiments, the secondary probe is a secondary clampFISH probe.

In some embodiments, methods of the present disclosure further comprise:
contacting the primary sample with (i) a set of secondary probes or (ii) a set of secondary probes and an agent that locks the set of tertiary probes to the primary probe, thereby forming a secondary sample;
contacting the secondary sample with (i) a set of primary probes or (ii) a set of primary probes and an agent that locks the set of primary probes to each secondary probe; and,
optionally, repeating the above steps until a desired level of fluorescent signal is achieved.

In some embodiments, methods of the present disclosure further comprise:
(g) contacting the secondary sample with (i) a set of tertiary probes or (ii) a set of tertiary probes and an agent that locks the set of tertiary probes to each secondary probe, thereby forming a tertiary sample;
(h) contacting the tertiary sample with (i) a set of secondary probes or (ii) a set of secondary probes and an agent that locks the set of secondary probes to each tertiary probe; and, optionally,
(i) repeating steps (g) and (h) until a desired level of fluorescent signal is achieved.

In some embodiments, the tertiary probe is a primary click-amplified Fluorescence In Situ Hybridization (clampFISH) probe, a Hybridization Chain Reaction (HCR) probe, or a Systematic Amplification of Bioorthogonal Reporting (SABER) probe. In some embodiments, the tertiary probe is a tertiary clampFISH probe.

In some embodiments, a primary probe is equivalent or similar in sequence (e.g., 100% sequence identity, 90% sequence identity, 80% sequence identity, 70% sequence identity, 60% sequence identity, 50% sequence identity, 40% sequence identity, etc.) and/or structure to a secondary or tertiary probe. In some embodiments, a primary probe is different in sequence (i.e., not 100% sequence identity) and/or structure to a secondary or tertiary probe. In some embodiments, a secondary probe is equivalent or similar in sequence (e.g., 100% sequence identity, 90% sequence identity, 80% sequence identity, 70% sequence identity, 60% sequence identity, 50% sequence identity, 40% sequence identity, etc.) and/or structure to a tertiary probe. In some embodiments, a secondary probe is different in sequence (i.e., not 100% sequence identity) and/or structure to a tertiary probe.

In some embodiments, the signal of the primary, secondary, and/or tertiary probes is detected by fluorescence. In some embodiments, the signal of the primary, secondary, and/or tertiary probes is detected by fluorescent in situ hybridization (FISH). In some embodiments, the signal of the primary, secondary, and tertiary clampFISH probes is detected by fluorescent in situ hybridization (FISH).

In some embodiments, a cell is a eukaryotic cell (e.g., mammalian cell). In some embodiments, the cell is attached to a surface (e.g., a glass slide or a petri dish) or suspended in liquid solution.

In some embodiments, isolating a nucleus of a cell comprises contacting the cell with a lysis buffer.

In some embodiments, the lysis buffer comprises a surfactant. In some embodiments, the surfactant is polysorbate, magnesium stearate, sodium dodecyl sulfate, TRITON™ N-101, TRITON™ X-100, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In some embodiments, the surfactant includes, but is not limited to: (i) cationic surfactant such as; cetyltrimethylammonium chloride, hexadecyltrimethylammonium chloride, benzalkonium chloride, benzethonium chloride, dioctadecyldimethylammonium bromide; (ii) anionic surfactant such as magnesium stearate, sodium dodecyl sulfate, dioctyl sodium sulfosuccinate, sodium myreth sulfate, perfluorooctanesulfonate, alkyl ether phosphates; (iii) non-ionic surfactant such as alkylphenol ethoxylates (Triton™ X-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactant such as cocamidopropyl hydroxysultaine, and 3-[(3-Cholamidopropyl) dimethylammonio]-1-propanesulfonate (CHAPS). In some embodiments, the surfactant is an alkylaryl polyether alcohol. In some embodiments, the surfactant is polyethylene glycol tert-octylphenyl ether.

In some embodiments, isolating a nucleus of a cell further comprises applying a mechanical force to the cell. In some embodiments, applying a mechanical force comprises using one or more of: mortar and pestle (e.g., using a mortar and pestle to ground the cell about 20-30 times), homogenization (e.g., grinding or shearing cells using manual or motorized homogenizers to release cellular contents), bead milling (e.g., utilizing small beads that agitate and rupture cells through rapid collision), ultrasonication (e.g., using high-frequency sound waves to generate cavitation and shear forces that break cell membranes), freeze-thaw (e.g., freezing the cells, causing ice crystals to rupture membranes, followed by thawing to release the contents), glass bead disruption (e.g., mixing cells with small beads and agitates them to physically break open the cell walls), and lyophilization (e.g., freezing and drying the cells, followed by mechanical grinding to disrupt the cell structure). In some embodiments, applying a mechanical force to a cell comprises using a mortar and pestle to ground the cell (e.g., about 20-30 times).

In some embodiments, the cell is contacted with the lysis buffer at from about 0° C. to about 25° C. (e.g., about 0° C. to about 20° C., about 0° C. to about 15° C., about 0° C. to about 14° C., about 0° C. to about 13° C., about 0° C. to about 12° C., about 0° C. to about 11° C., about 0° C. to about 10° C., about 0° C. to about 9° C., about 0° C. to about 8° C., about 0° C. to about 7° C., about 0° C. to about 6° C., about 0° C. to about 5° C., etc.). In some embodiments, isolating a nucleus of a cell further comprises contacting the cell with the lysis buffer at about 4° C.

In some embodiments, the cell is contacted with the lysis buffer for from about 0 minutes to about 30 minutes (e.g., about 0 minutes to about 25 minutes, about 0 minutes to about 20 minutes, about 0 minutes to about 15 minutes, about 5 minutes to about 15 minutes, etc.). In some embodiments, isolating a nucleus of a cell further comprises contacting the cell with the lysis buffer for about 10 minutes.

In some embodiments, the cell is contacted with the lysis buffer at about 4° C. for about 10 minutes.

The present disclosure includes the use of any compositions and methods for crosslinking or non-crosslinking fixation known in the art. In some embodiments, a sample (e.g., a cell, a nucleus) is fixed using an aldehyde-based fixative. For example, in some embodiments, methods of the present disclosure comprise fixing a sample using a fixative comprising formaldehyde or paraformaldehyde. In some embodiments, a sample is fixed using an alcohol-based fixative (e.g., a fixative comprising methanol or ethanol). Fixation of a sample may be done under any suitable conditions which results in the fixation of the sample. For example, in one embodiment, a sample is contacted with a fixative for about 1 second to about 5 hours. In one embodiment, a sample is contacted with a non-cross-linking fixative (e.g., methanol) for about 1 minute. In another embodiment, a sample is contacted with a non-crosslinking fixative for about 10 minutes. In one embodiment, the sample is contacted with a non-crosslinking fixative for about 1 to about 10 minutes and any and all ranges therebetween. In one embodiment, a sample is contacted with the fixative at a temperature of about −80° C. to about 50° C. In some embodiments, a sample is contacted with a non-crosslinking fixative at a temperature of about −20° C. In other embodiments, a sample is contacted with a crosslinking fixative at room temperature (e.g., about 20-23° C.). In one embodiment, following incubation with a fixative, a sample is washed.

In some embodiments, a fixative comprises dithiobis(succinimidyl propionate) (DSP) or disuccinimidyl sulfoxide (DSS), formaldehyde, or a combination thereof. In some embodiments, a fixative comprises dithiobis(succinimidyl propionate) (DSP) or disuccinimidyl sulfoxide (DSS). In some embodiments, a fixative comprises dithiobis(succinimidyl propionate) (DSP).

In some embodiments, a fixative (e.g., dithiobis(succinimidyl propionate) or disuccinimidyl sulfoxide) has a concentration of from about 0 μM to about 1000 μM (e.g., about 0 μM to about 1000 μM, about 0 μM to about 900 μM, about 0 μM to about 800 μM, about 100 μM to about 800 μM, about 200 μM to about 800 M, about 200 μM to about 700 μM, about 300 μM to about 700 μM, 300 μM to about 600 μM, 400 μM to about 600 μM, etc.). In some embodiments, a fixative (e.g., dithiobis(succinimidyl propionate) or disuccinimidyl sulfoxide) has a concentration of about 500 μM.

In some embodiments, a nucleus is contacted with a fixative for from about 0 minutes to about 30 minutes (e.g., about 0 minutes to about 25 minutes, about 0 minutes to about 20 minutes, about 0 minutes to about 15 minutes, about 5 minutes to about 15 minutes, etc.). In some embodiments, a nucleus is contacted with a fixative for about 10 minutes.

In some embodiments, a fixed nucleus is contacted with a surfactant (e.g., a first surfactant) for from about 1 minute to about 30 minutes. In some embodiments, a fixed nucleus is contacted with a surfactant for from about 5 minutes to about 30 minutes. In some embodiments, contacting the fixed nucleus with a surfactant comprises contacting (e.g., incubating) the fixed nucleus with the surfactant (e.g., 1×PBS/0.1% w/v Triton™ X-100) for from about 1 minute to about 10 minutes (e.g., about 5 minutes) twice. In some embodiments, the surfactant (e.g., a first surfactant) is an alkylaryl polyether alcohol (e.g., polyethylene glycol tert-octylphenyl ether). In some embodiments, the concentration of the alkylaryl polyether alcohol in the surfactant (e.g., a first surfactant) is 0.1% w/v. In some embodiments, the surfactant is polyethylene glycol tert-octylphenyl ether.

In some embodiments, methods of the present disclosure further comprise contacting the fixed nucleus with a second surfactant. In some embodiments, methods of the present disclosure further comprise contacting (e.g., incubating) the fixed nucleus with a second surfactant for about 20 minutes. In some embodiments, In some embodiments, the surfactant (e.g., a second surfactant) is an alkylaryl polyether alcohol (e.g., polyethylene glycol tert-octylphenyl ether). In some embodiments, the concentration of the alkylaryl polyether alcohol in the surfactant (e.g., a second surfactant) is 0.25% w/v. In some embodiments, the surfactant is polyethylene glycol tert-octylphenyl ether.

In some embodiments, a surfactant is polysorbate, magnesium stearate, sodium dodecyl sulfate, Triton™ X-100, Triton™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In some embodiments, the surfactant includes, but is not limited to: (i) cationic surfactant such as; cetyltrimethylammonium chloride, hexadecyltrimethylammonium chloride, benzalkonium chloride, benzethonium chloride, dioctadecyldimethylammonium bromide; (ii) anionic surfactant such as magnesium stearate, sodium dodecyl sulfate, dioctyl sodium sulfosuccinate, sodium myreth sulfate, perfluorooctanesulfonate, alkyl ether phosphates; (iii) non-ionic surfactant such as alkylphenol ethoxylates (e.g., Triton™ X-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactant such as cocamidopropyl hydroxysultaine, and 3-[(3-Cholamidopropyl) dimethylammonio]-1-propanesulfonate (CHAPS). In some embodiments, a surfactant is a non-ionic surfactant. In some embodiments, the surfactant is an alkylaryl polyether alcohol. In some embodiments, the surfactant is polyethylene glycol tert-octylphenyl ether.

In some embodiments, hybridizing a primary probe to nuclear RNA comprises contacting the nucleus with the primary probe. For example, in an embodiment, hybridizing a primary probe to nuclear RNA comprises contacting the nucleus with a hybridization solution comprising the primary probe. In some embodiments, hybridizing a primary probe to nuclear RNA comprises contacting the nucleus with the primary probe for at least about 1 hour (e.g., at least about 1.5 hours, at least about 2 hours, at least about 2.5 hours, at least about 3 hours, at least about 3.5 hours, at least about 4 hours, at least about 4.5 hours, at least about 5 hours, etc.). In some embodiments, hybridizing a primary probe to nuclear RNA comprises contacting the nucleus with the primary probe for from about 4 hours to about 6 hours. In some embodiments, hybridizing a primary probe to nuclear RNA comprises contacting the nucleus with the primary probe for about 4 hours.

In some embodiments, hybridizing a primary clampFISH probe to nuclear RNA comprises contacting the nucleus with the primary clampFISH probe. For example, in an embodiment, hybridizing a primary clampFISH probe to nuclear RNA comprises contacting the nucleus with a hybridization solution comprising the primary clampFISH probe. In some embodiments, hybridizing a primary clampFISH probe to nuclear RNA comprises contacting the nucleus with the primary clampFISH probe for at least about 1 hour (e.g., at least about 1.5 hours, at least about 2 hours, at least about 2.5 hours, at least about 3 hours, at least about 3.5 hours, at least about 4 hours, at least about 4.5 hours, at least about 5 hours, etc.). In some embodiments, hybridizing a primary clampFISH probe to nuclear RNA comprises contacting the nucleus with the primary clampFISH probe for from about 4 hours to about 6 hours. In some embodiments, hybridizing a primary clampFISH probe to nuclear RNA comprises contacting the nucleus with the primary clampFISH probe for about 4 hours.

In some embodiments, hybridizing a primary probe to nuclear RNA comprises contacting the nucleus with the primary probe at from about 10° C. to about 50° C. (e.g., about 15° C. to about 50° C., about 15° C. to about 45° C., about 20° C. to about 45° C., about 20° C. to about 40° C., about 25° C. to about 40° C., about 30° C. to about 40° C., about 35° C. to about 40° C., etc.). In some embodiments, hybridizing a primary probe to nuclear RNA comprises contacting the nucleus with the primary probe at about 37° C.

In some embodiments, hybridizing a primary clampFISH probe to nuclear RNA comprises contacting the nucleus with the primary clampFISH probe at from about 10° C. to about 50° C. (e.g., about 15° C. to about 50° C., about 15° C. to about 45° C., about 20° C. to about 45° C., about 20° C. to about 40° C., about 25° C. to about 40° C., about 30° C. to about 40° C., about 35° C. to about 40° C., etc.). In some embodiments, hybridizing a primary clampFISH probe to nuclear RNA comprises contacting the nucleus with the primary clampFISH probe at about 37° C.

In some embodiments, hybridizing a primary probe to nuclear RNA comprises contacting the nucleus with the primary probe for at least about 4 hours at about 37° C.

In some embodiments, hybridizing a primary clampFISH probe to nuclear RNA comprises contacting the nucleus with the primary clampFISH probe for at least about 4 hours at about 37° C.

In some embodiments, the primary probe (e.g., primary clampFISH probe) has a concentration of about 1 μM to about 100 mM (e.g., about 10 μM to about 10 mM, about 100 μM to about 1 mM, about 200 μM to about 500 μM, about 400 μM) in a hybridization solution. The hybridization solution may further comprise any additional suitable components known in the art. For example, in one embodiment, the hybridization solution comprises formamide, saline-sodium citrate, and dextran sulfate.

In some embodiments, a sample (e.g., a primary sample) is contacted with an agent. In some embodiments, a sample (e.g., a primary sample) is contacted with an agent for at least about 1 minute (e.g., at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, etc.). In some embodiments, a sample (e.g., a primary sample) is contacted with an agent for from about 10 minutes to about 30 minutes. In some embodiments, a sample (e.g., a primary sample) is contacted with an agent for about 10 minutes.

In some embodiments, a sample (e.g., a primary sample) is contacted with an agent from about 10° C. to about 50° C. (e.g., about 15° C. to about 50° C., about 15° C. to about 45° C., about 20° C. to about 45° C., about 20° C. to about 40° C., about 25° C. to about 40° C., about 30° C. to about 40° C., about 35° C. to about 40° C., etc.). In some embodiments, a sample (e.g., a primary sample) is contacted with an agent at about 37° C.

In some embodiments, a sample (e.g., a primary sample) is contacted with an agent for at least about 10 minutes at about 37° C.

In some embodiments, an agent is a click chemistry agent. In some embodiments, an agent is a click chemistry agent catalyzing a cycloaddition, thereby locking the probe (e.g., a primary probe, a secondary probe, a tertiary probe) to the nuclear RNA. In some embodiments, the agent is a click chemistry agent catalyzing a cycloaddition between 5′ and 3′ end of a probe (e.g., a primary probe, a secondary probe, a tertiary probe), thereby locking the probe to the nuclear RNA. In some embodiments, an agent may comprise a metal (e.g., a metal ion, a metal salt). In some embodiments, an agent comprises copper (I), copper (II) or a ruthenium. In some embodiments, an agent comprises copper.

In some embodiments, the copper in the Cu (I) reduction state. In some embodiments, the copper can be provided in the Cu (II) reduction state (e.g., as a salt, such as but not limited to Cu(NO₃)₂Cu(OAc)₂ or CuSO₄), in the presence of a reducing agent wherein Cu (I) is formed in situ by the reduction of Cu (II). Such reducing agents include, but are not limited to, ascorbate, Tris(2-Carboxyethyl) Phosphine (TCEP), 2,4,6-trichlorophenol (TCP), NADH, NADPH, thiosulfate, metallic copper, quinone, hydroquinone, vitamin K₁, glutathione, cysteine, 2-mercaptoethanol, dithiothreitol, Fe²⁺, Co²⁺, or an applied electric potential. In other embodiments, the reducing agents include metals selected from Al, Be, Co, Cr, Fe, Mg, Mn, Ni, Zn, Au, Ag, Hg, Cd, Zr, Ru, Fe, Co, Pt, Pd, Ni, Rh, and W. In other embodiments, the copper is in the Cu (II) state and is reduced to Cu (I) with sodium ascorbate.

In some embodiments, contacting a sample (e.g., a primary sample, a secondary sample, a tertiary sample) with an agent is performed in water and a variety of solvents, including mixtures of water and a variety of (partially) miscible organic solvents including alcohols, dimethyl sulfoxide (DMSO), dimethyl formamide (DMF), tert-butanol (tBuOH) and acetone.

Certain metal ions may be unstable in aqueous solvents, e.g., Cu (I), therefore stabilizing ligands/chelators may be used. In some embodiments, an agent further comprises at least one metal chelator (e.g., copper chelator). In some embodiments, such chelators bind copper in the Cu (I) state. In some embodiments, at least one copper chelator is used in the methods described herein. In some embodiments, the copper (I) chelator is a 1,10 phenanthroline-containing copper (I) chelator. Non-limiting examples of such phenanthroline-containing copper (I) chelators include, but are not limited to, bathophenanthroline disulfonic acid (4,7-diphenyl-1,10-phenanthroline disulfonic acid) and bathocuproine disulfonic acid (BCS; 2,9-dimethyl-4,7-diphenyl-1,10-phenanthroline disulfonate). Other chelators used in such methods include, but are not limited to, N-(2-acetamido) iminodiacetic acid (ADA), pyridine-2,6-dicarboxylic acid (PDA), S-carboxymethyl-L-cysteine (SCMC), trientine, tetra-ethylenepolyamine (TEPA), NNNN-tetrakis(2-pyridylmethyl)ethylenediamine (TPEN), EDTA, neocuproine, N-(2-acetamido) iminodiacetic acid (ADA), pyridine-2,6-dicarboxylic acid (PDA), S-carboxymethyl-L-cysteine (SCMC), tris-(benzyl-triazolylmethyl)amine (TBTA), or a derivative thereof. Most metal chelators, a wide variety of which are known in the art, are known to chelate several metals, and thus metal chelators in general may be tested for their function in 1,3 cycloaddition reactions catalyzed by copper. In some embodiments, histidine is used as a chelator, while in other embodiments glutathione is used as a chelator and a reducing agent.

In some embodiments, an agent further comprises a reducing agent. The concentration of the reducing agent may be in the micromolar to millimolar range. In some embodiments, the concentration of the reducing agent is from about 100 μM to about 100 mM. In other embodiments, the concentration of the reducing agent is from about 10 μM to about 10 mM. In other embodiments, the concentration of the reducing agent is from about 1 μM to about 1 mM. In yet other embodiments, the concentration of the reducing agent is 2.5 mM.

In some embodiments, the concentration of chelator is in the micromolar to millimolar range, e.g., from 1 μM to 100 mM. In some embodiments, the chelator concentration is from about 10 μM to about 10 mM. In other embodiments, the chelator concentration is from about 50 μM to about 10 mM. In other embodiments, the chelator may be provided in a solution that includes a water miscible solvent such as, alcohols, dimethyl sulfoxide (DMSO), dimethyl formamide (DMF), tert-butanol (tBuOH) and acetone. In other embodiments, the chelator may be provided in a solution that includes a solvent such as, for example, dimethyl sulfoxide (DMSO) or dimethylformamide (DMF).

In some embodiments, an agent (e.g., a click chemistry agent) catalyzes an azide-alkyne cycloaddition, thereby locking the primary, secondary and tertiary probes to their respective nucleic acid target. In some embodiments, an agent (e.g., a click chemistry agent) connects the azide and alkyne groups, located at the 3′ and 5′ ends of the primary, secondary and tertiary probes around their respective nucleic acid target. In some embodiments, an agent (e.g., a click chemistry agent) connects the azide and alkyne groups, located internally within the primary, secondary and tertiary probes, around their respective nucleic acid target. Click chemistry, as described in further details below herein, is a cycloaddition well known in the art where pairs of functional groups (e.g. alkyne and an azide) rapidly and selectively react and couple with each other. In some embodiments, a click chemistry reaction is catalyzed by a metal agent. In some embodiments, a click chemistry reaction is catalyzed by copper (I), a copper (II) or a ruthenium. In some embodiments, an agent (e.g., a click chemistry agent) comprises a copper (II), BTTAA ligand, and sodium ascorbate. In other embodiments, a click chemistry reaction is a copper-free strain promoted azide alkyne cycloaddition (SpAAC) comprising a cyclooctyne that undergoes azide-alkyne Huisgen cycloaddition without the need of a copper catalyzer.

### Probes

In some embodiments, hybridizing a primary probe to nuclear RNA comprises contacting a fixed nucleus with one or more primary probes. In some embodiments, the one or more primary probes are contacted with a set of secondary probes, wherein the secondary probes bind in 2:1 ratio to the one or more primary probes. In other embodiments, the one or more primary probes are contacted with a set of secondary probes, wherein the secondary probes bind in a n: 1 ratio to each of the one or more primary probes, wherein “n” corresponds to the number of binding sites on each of the one or more primary probes. In some embodiments, the secondary probes are in turn contacted with a set of tertiary probes, wherein the tertiary probes bind in 2:1 ratio to the secondary probes. In yet another embodiment, the secondary probes are contacted with a set of tertiary probes, wherein the tertiary probes bind in a n: 1 ratio to the secondary probes, wherein “n” corresponds to the number of binding sites on the secondary probe. In other embodiments, the tertiary probes are contacted with a set of secondary probes, wherein the secondary probes bind in 2:1 ratio to the tertiary probes. In further embodiments, the tertiary probes are contacted with a set of secondary probes, wherein the secondary probes bind in a n: 1 ratio to the tertiary probes, wherein “n” corresponds to the number of binding sites on the tertiary probes. In some embodiments, the steps involving the contacting of the secondary or tertiary probes are repeated (e.g., these steps are repeated for about 1 to about 20 or more rounds). The number of rounds may be selected by the user based upon the type of sample and the desired level of signal amplification.

In some embodiments, a desired level of fluorescent signal is achieved such that the level of fluorescent signal of the primary probe is amplified. In some embodiments, the fluorescent signal is amplified more than about 10, more than about 50, more than about 100 and more than about 500 fold (i.e., times) as compared to a control labeled with a standard FISH or labeled with a primary, secondary and tertiary probes but without repeating any of the above steps involving the contacting of the secondary or tertiary probes. In some embodiments, the signal is amplified about 4 fold per 2 rounds. In other embodiments, the fluorescent signal is amplified about 120 fold after 6 rounds, and about 500 fold after 10 rounds.

In some embodiments, the desired level of fluorescent signal is more than about 2 (e.g., more than about 5, more than about 10, more than about 50, more than about 100, more than about 500 fold) as compared to a control labeled with a standard FISH or labeled with a primary, secondary and/or tertiary probes. In some embodiments, the desired level of fluorescent signal is about 4 fold. In other embodiments, the desired level of fluorescent signal is about 120 fold or about 500 fold.

Probes of the present disclosure may be DNA, RNA or mixtures of DNA and RNA. They may include non-natural nucleotides, and they may include non-natural internucleotide linkages. Non-natural nucleotides that increase the binding affinity of probes include 2′-O-methyl ribonucleotides, for example.

In some embodiments, a probe (e.g., a primary, secondary, tertiary probe) comprises about 40 to about 300 nucleotides (e.g., about 100 to about 200 nucleotides, about 125 to about 175 nucleotides, etc.). In some embodiments, a probe comprises about 150 nucleotides. In some embodiments, a probe comprises a left binding arm, a left adapter, a backbone, a right adapter and a right binding arm. In some embodiments, a binding arm (e.g., left binding arm, right binding arm) comprises about 15 nucleotides and an adapter (e.g., left adapter, right adapter) comprises about 10 nucleotides. In some embodiments, a binding arm (e.g., left binding arm, right binding arm) comprises about 25 nucleotides and an adapter (e.g., left adapter, right adapter) comprises about 20 nucleotides. In some embodiments, the backbone comprises from about 90 to about 100 nucleotides. In some embodiments, the backbone comprises about 90 nucleotides. In some embodiments, the backbone comprises about 92 nucleotides. In some embodiments, the backbone comprises about 100 nucleotides.

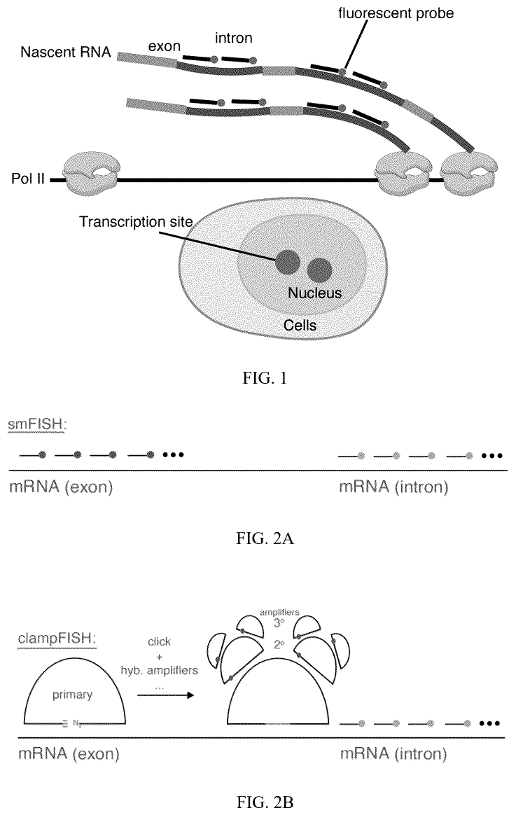

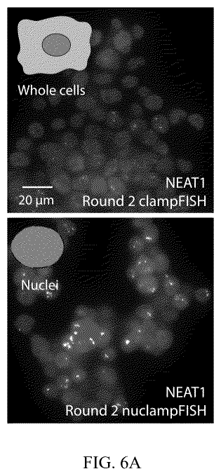

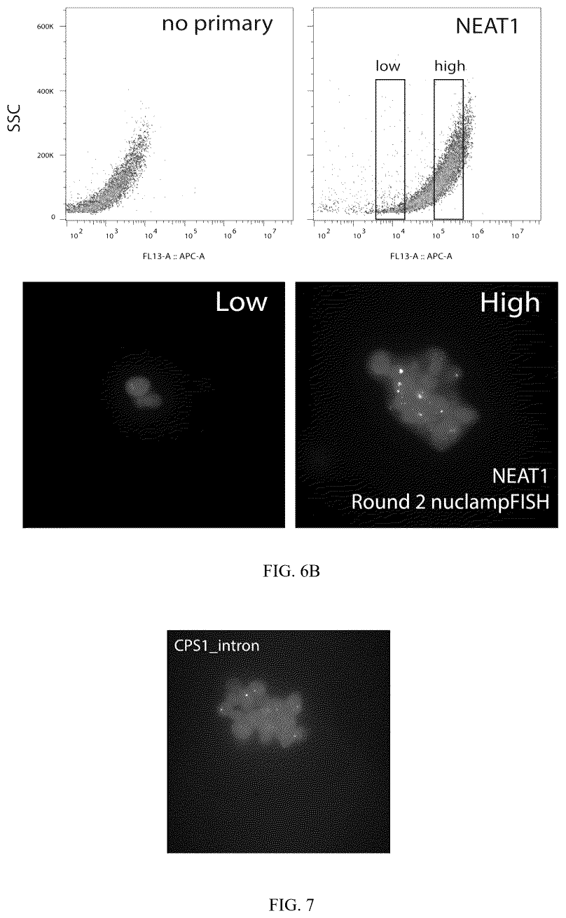

Examples of clampFISH probes are disclosed in FIGS. 1C, 6A, and 6B of US. Pub. No. US 2019/0382838 A1, which is incorporated by reference herein in its entirety.

In one embodiment, the nuclear RNA is targeted by one or more probe sets comprising one or more probes, each targeting a region of the nuclear RNA. In some embodiments, the targeted region is 30 nucleotides long and is bound by 2 adjacent 15 nucleotides long binding arms (e.g., left and right arms). In some embodiments, the targeted region is 30 nucleotides long and is bound by 2 adjacent 25 nucleotides long binding arms (e.g., left and right arms). In other embodiments, the target region is larger or smaller than 30 nucleotides long (e.g., 14 nucleotides long) and is bound by 2 adjacent binding arms having a total length equal or less to the target region (e.g., 7 nucleotides long left and right binding arms targeting a 14 nucleotides long region). In some embodiments, each probe of the primary, secondary and tertiary probes binds to a different region of their respective nucleic acid target. In some embodiments, the primary, secondary and tertiary probes are complementary through their 3′ and 5′ ends (e.g., binding arms) to two regions of their respective nucleic acid target.

In some embodiments, the 3′ and 5′ ends of a primary, secondary, and/or tertiary probe comprise a binding arm comprising about 25 nucleotides. In some embodiments, the 3′ and 5′ ends of a primary, secondary, and tertiary probe comprise a binding arm comprising about 25 nucleotides. In some embodiments, the 3′ and 5′ ends of a primary, secondary, and/or tertiary probe comprise a binding arm comprising about 15 nucleotides. In some embodiments, the 3′ and 5′ ends of a primary, secondary, and tertiary probe comprise a binding arm comprising about 15 nucleotides.

In a non-limiting example, EEF2 clampFISH probes (e.g., probes listed in Table 4) may be hybridized to EEF2 mRNA using a backbone sequence. In some embodiments, the signal is detected using fluorophore (e.g., Cy5, alexa, etc.) built into a region (e.g., left binding arm, a left adapter, a backbone, a right adapter and a right binding arm) of a primary probe, secondary probe, tertiary probe, or a combination thereof. In some embodiments, the signal is detected using a Cy5 fluorophore built into the left arm sequence of a secondary probe and a tertiary probe.

In some embodiments, the 3′ and 5′ ends of the probes comprise an azide and an alkyne group respectively. In other embodiments, the 3′ and 5′ ends of the probes comprise an alkyne and an azide group respectively. In other embodiments, the internal region of the probes comprises at two separate locations an azide and an alkyne group. In some embodiments, an agent (e.g., click chemistry agent) catalyzes an azide-alkyne cycloaddition thereby covalently locking the primary, secondary and tertiary probes around their respective nucleic acid target. In some embodiments, an agent (e.g., click chemistry agent) connects the azide and alkyne groups, located at the 3′ and 5′ ends of the primary, secondary and tertiary probes around their respective nucleic acid target.

In some embodiments, probes are used for a multiplex assay. In certain embodiments, more than one type of probe is used. For example, in certain embodiments, about 1 to about 1000 different probes are used. In one embodiment, each of the different probes are labeled with a similar of different fluorophore and are hybridized simultaneously to a target sequence of a nucleotide molecule, such as an RNA molecule. In some embodiments, the probes are not labeled and comprise unique backbone regions that bind secondary and tertiary probes. In some embodiments, a terminating probe (e.g. a tertiary probe) is labeled with a fluorophore, e.g., using techniques used in single molecule fluorescent in situ hybridization (smFISH). In certain embodiments, the number of probes used for a singleplex or multiplex assay ranges from about 4 to about 100, from about 10 to about 80, from about 15 to about 70, or from about 20 to about 60. A fluorescent spot is created that can be detected from the combined fluorescence of the multiple probes. The probes may be non-overlapping, i.e., the region of the target sequence to which each probe hybridizes is unique (or at least non-overlapping). Probes in a set of 2 or more for a selected target sequence can be designed to hybridize adjacently to one another or to hybridize non-adjacently, with stretches of the target sequence, from one nucleotide to a hundred nucleotides or more, not complementary to any of the probes.

In some embodiments, a single cell can be probed simultaneously for multiple nucleic acid (e.g., nuclear RNA) target sequences, either more than one target sequence of one nucleic acid molecule, or one or more sequences of different nucleic acid molecules. Additionally, one target sequence of a nucleic acid molecule (e.g., nuclear RNA) may be probed with more than one set of probes, wherein each set is labeled with a distinguishable fluorophore, and the fluorophores are distinguishable.

Methods of the present disclosure may also include determining if one or more fluorescent signal spots representing a target sequence (e.g., nuclear RNA sequence) is present. Methods according to the present disclosure also allow counting fluorescent signal spots of a given color corresponding to a given nucleic acid species. When it is desired to detect more than one nucleic acid species, different sets of probes labeled with distinct fluorophores can be used in the same hybridization mixture. Fluorescent signal of the fluorophores may be detected by using any microscopic or flow cytometry methods known in the art.

In some embodiments, a probe (e.g., primary, secondary, tertiary HCR probe) is a DNA hairpin.

In some embodiments, a probe (e.g., primary, secondary, tertiary clampFISH probe) is a DNA probe labeled with a fluorophore. The fluorophore may be attached at any position, including, without limitation, attaching a fluorophore to one end of a probe, preferably to the 3′ end. The probes may be included in a hybridization solution that contains the probes in excess.

In some embodiments, the nuclear RNA is pre-mRNA, ribosomal RNA (rRNA), transfer RNA (tRNA), small nuclear RNA (snRNA), or a combination thereof. In some embodiments, the nuclear RNA is pre-mRNA.

The present disclosure also relates to, in part, methods of detecting nuclear RNA (e.g., nuclear RNA from a subject), the methods comprising:
(a) isolating a nucleus of a cell (e.g., a cell from a subject, a mammalian cell), the nucleus comprising a nuclear RNA;
(b) contacting the nucleus with a fixative, thereby producing a fixed nucleus;
(c) contacting the fixed nucleus with a surfactant;
(d) hybridizing a primary probe to the nuclear RNA, thereby forming a primary sample comprising labeled nuclear RNA; and
(e) detecting the labeled nuclear RNA.

In some embodiments, methods of detecting nuclear RNA (e.g., nuclear RNA from a subject) comprise:
(a) isolating a nucleus of a cell (e.g., a cell from a subject, a mammalian cell), the nucleus comprising a nuclear RNA;
(b) contacting the nucleus with a fixative, thereby producing a fixed nucleus;
(c) contacting the fixed nucleus with a surfactant;
(d) hybridizing a primary probe to the nuclear RNA, thereby forming a primary sample comprising labeled nuclear RNA;
(e) contacting the primary sample with an agent that locks the primary probe to the nuclear RNA; and
(f) detecting the labeled nuclear RNA.

In some embodiments, detecting labeled nuclear RNA comprises using one or more of: flow cytometry, Northern blotting, Quantitative PCR (qPCR), RNA sequencing (RNA-Seq), and microscopy (e.g., in situ hybridization, expansion microscopy, etc.). In some embodiments, detecting labeled nuclear RNA comprises using flow cytometry, microscopy, or a combination thereof.

In some embodiments, detecting labeled nuclear RNA comprises using microscopy (e.g., low magnification microscopy) at a magnification of about 60× or less, about 40× or less, about 20× or less, about 10× or less, or about 4× or less.

In some embodiments, methods of the present disclosure are used in conjunction with expansion microscopy. Expansion microscopy is a method well known in the art where a sample (e.g., a cell) is linked to a swellable polymer and physically expanded to enable a high-resolution microscopy using a low magnification microscope (U.S. patent application Ser. No. 15/098,799).

In some embodiments, prior to detecting the labeled nuclear RNA and after contacting the primary sample with an agent that locks the primary probe to the nuclear RNA, methods of the present disclosure may further comprise one or more of:
(i) contacting the primary sample with a set of secondary probes or a set of secondary probes and an agent that locks the set of secondary probes to the primary probe, thereby forming a secondary sample;
(ii) contacting the secondary sample with a set of tertiary probes or a set of tertiary probes and an agent that locks the set of tertiary probes to each secondary probe, thereby forming a tertiary sample;
(iii) contacting the tertiary sample with a set of secondary probes or a set of tertiary probes and an agent that locks the set of secondary probes to each tertiary probe; and
(iv) repeating steps (ii) and (iii) until a desired level of fluorescent signal is achieved.

In some embodiments disclosed herein are methods of analyzing nuclear RNA (e.g., nuclear RNA from a subject), the methods comprising:
(a) isolating a nucleus of a cell (e.g., a cell from a subject, a mammalian cell), the nucleus comprising nuclear RNA;
(b) contacting the nucleus with a fixative, thereby producing a fixed nucleus;
(c) contacting the fixed nucleus with a surfactant;
(d) hybridizing a primary probe to the nuclear RNA, thereby forming a primary sample comprising labeled nuclear RNA;
(e) detecting the nuclear RNA;
(f) contacting the fixed nucleus with a reducing agent, thereby unfixing the nucleus; and
(g) analyzing the nuclear RNA.

In some embodiments, methods of analyzing nuclear RNA (e.g., nuclear RNA from a subject) comprise:
(a) isolating a nucleus of a cell (e.g., a cell from a subject, a mammalian cell), the nucleus comprising nuclear RNA;
(b) contacting the nucleus with a fixative, thereby producing a fixed nucleus;
(c) contacting the fixed nucleus with a surfactant;
(d) hybridizing a primary probe to the nuclear RNA, thereby forming a primary sample comprising labeled nuclear RNA;
(e) contacting the primary sample with an agent that locks the primary probe to the nuclear RNA;
(f) detecting the nuclear RNA;
(g) contacting the fixed nucleus with a reducing agent, thereby unfixing the nucleus; and
(h) analyzing the nuclear RNA.

In some embodiments, the reducing agent is dithiothreitol (DTT) or tris(2-carboxyethyl) phosphine (TCEP). In some embodiments, the concentration of the reducing agent is from about 0.1 mM to about 500 mM (e.g., about 1 mM to about 500 mM, about 1 mM to about 400 mM, about 1 mM to about 300 mM, about 1 mM to about 200 mM, about 1 mM to about 100 mM, about 1 mM to about 90 mM, about 1 mM to about 80 mM, about 1 mM to about 70 mM, about 1 mM to about 60 mM, about 1 mM to about 50 mM, about 5 mM to about 50 mM, about 10 mM to about 50 mM, about 20 mM to about 30 mM, etc.). In some embodiments, the fixed nucleus is contacted with a reducing agent with a concentration of from about 10 to about 50 mM. In some embodiments, the fixed nucleus is contacted with a reducing agent with a concentration of about 25 mM.

In some embodiments, a fixed nucleus is contacted with a reducing agent with a pH of from about 8.0 to about 9.0 (e.g., about 8.5 to about 9.0). In some embodiments, the pH is about 8.5.

In some embodiments, a fixed nucleus is contacted with a reducing agent for at least about 1 minute (e.g., at least about 5 minutes, at least about 10 minutes, at least about 20 minutes, at least about 25 minutes, etc.). In some embodiments, a fixed nucleus is contacted with a reducing agent for about 30 minutes.

In some embodiments, a fixed nucleus is contacted with a reducing agent at from about 10° C. to about 50° C. (e.g., about 15° C. to about 50° C., about 15° C. to about 45° C., about 20° C. to about 45° C., about 20° C. to about 40° C., about 25° C. to about 40° C., about 30° C. to about 40° C., about 35° C. to about 40° C., etc.). In some embodiments, a fixed nucleus is contacted with a reducing agent at about 37° C.

In some embodiments, a fixed nucleus is contacted with about 25 mM DTT at about 37° C. for about 30 minutes.

In some embodiments, analyzing nuclear RNA comprises using mass spectrometry (MS) (e.g., tandem MS, Electrospray Ionization Mass Spectrometry (ESI)-MS, Matrix-Assisted Laser Desorption/Ionization (MALDI)-MS, etc.), a biochemical assay (e.g., Electrophoretic Mobility Shift Assay (EMSA), RNA pulldown assays, splicing assays, RNA ligase assays, RNA helicase assays, etc.), chromatin profiling (e.g., chromatin immunoprecipitation, sequencing, chromatin immunoprecipitation-RNA, assay for transposase-accessible chromatin with sequencing, etc.), or a combination thereof. In some embodiments, analyzing nuclear RNA comprises using mass spectrometry.

In some embodiments, analyzing nuclear RNA comprises identifying a splice junction in the nuclear RNA. In some embodiments, each arm of the primary probe targets the nucleic acid region closest to the respective exons. In other embodiments, analyzing labeled nuclear RNA comprises identifying alternatively spiced variants. In other embodiments, analyzing labeled nuclear RNA comprises identifying a mutation in the nuclear RNA.

In some embodiments, prior to analyzing the labeled nuclear RNA and after contacting the primary sample with an agent that locks the primary probe to the nuclear RNA, methods of the present disclosure may further comprise one or more of:
(i) contacting the primary sample with a set of secondary probes or a set of secondary probes and an agent that locks the set of secondary probes to the primary probe, thereby forming a secondary sample;
(ii) contacting the secondary sample with a set of tertiary probes or a set of tertiary probes and an agent that locks the set of tertiary probes to each secondary probe, thereby forming a tertiary sample;
(iii) contacting the tertiary sample with a set of secondary probes or a set of tertiary probes and an agent that locks the set of secondary probes to each tertiary probe; and
(iv) repeating steps (ii) and (iii) until a desired level of fluorescent signal is achieved.

In some embodiments, the present disclosure relates to the nuclampFISH method for targeting nuclear RNA and transcription sites with a reversible crosslinker. The nuclampFISH amplification strategy is used to sort cells for the first time based on transcriptional activity. A chromatin accessibility assay is performed to understand the differences between transcriptionally active and inactive cells.

In one example embodiment, to enhance nuclear accessibility, a specific set of conditions are used:
1. the nuclei are separated from the cytoplasm
2. a high formamide concentration is used to reduce nonspecific binding
3. ethanol permeabilization is eliminated and replaced with a surfactant-based permeabilization
4. Standard formaldehyde crosslinking is replaced with reversible crosslinking (specifically DSP, but other reversible crosslinkers may also be used)
5. A high salt concentration is used in the hybridization step to drive binding to the specific target.
6. An increased number of probes is used to target.

Single-molecule fluorescent in situ hybridization is the gold standard for imaging and quantifying RNA molecules inside cells. However, the total fluorescence produced is insufficient for high-throughput methods. For example, separation of cells based on RNA expression using flow cytometry or high throughput imaging using low magnification microscopy. To circumvent these challenges, FISH-based amplification strategies have been used to maintain the specificity but greatly enhance the signal. These methods include bDNA (e.g., RNAscope™), Hybridization chain reaction (HCR), and clampFISH. Each technique has difficulty accessing and amplifying nuclear RNAs, limiting the analysis of long non-coding RNAs and, significantly, transcription sites needed to track active RNA expression in cells.

Example embodiments of the invention are characterized by one or more of the following features: Amplification of signal from transcription sites; sorting cells based on transcriptional activity of a single gene; use of a reversible crosslinker enables biochemical analysis such as mass spectrometry from these samples after amplification; and sorting cells based on transcriptional activity of a specific gene.

Example embodiments of the invention are characterized by one or more of the following advantages: enhanced accessibility of probes to nuclear RNAs; signal enhancement for nuclear RNAs; and high specificity. Current methods cannot access nuclear RNAs for sufficient amplification.

Example embodiments of the invention are useful for more of the following: by one or more of the following: imaging-based profiling of single cells; sorting cells based on transcriptional activity; Single-molecule analysis within single cells; diagnosis.

In some embodiments, methods of the present disclosure are used in the analysis of fixed (not live) cells. In some embodiments, methods of the present disclosure are compatible with techniques such as immunocytochemistry, flow cytometry, and proteomic assays.

In some embodiments, methods of the present disclosure are used to determine whether therapy (e.g., gene therapy) is effective, on a single cell level. For example, the delivery of a therapeutic gene to individual cells may be assessed, as well as cellular responses to the therapy.

Example embodiments of the invention are useful for proteomic assays, e.g., genome-wide assays.

The growth of the market is attributed to factors, such as an increasing demand for in vitro diagnostics (IVD), used in the diagnosis of various chronic diseases aided by the high levels of reliability, rapidity, and sensitivity associated with the technique. Moreover, over the years, diagnostics based on DNA probes have gained immense acceptance in medical diagnostics for the detection of diseases caused by bacteria or pathogens owing to the rising need for providing effective and early treatment. For example, the transcriptional activation of cancer markers in patient tumor samples, across all types of cancer, would be potential diagnostic applications of this technique.

Disclosed herein, in part, are embodiments of nuclampFISH, an amplified FISH method for targeting nuclear RNA and transcription sites. NuclampFISH achieves the goal of specific detection of nuclear RNA (including transcription sites) and amplifying the FISH signal of nuclear RNA (including transcription sites). Based on the specific and amplified nuclampFISH signal, cells may be separated according to the expression level of transcription sites for downstream analysis to understand transcription better. FISH detection and chromatin analysis are combined with a chemical reversible crosslinker DSP.

When compared to clampFISH and HCR FISH for transcription sites, nuclampFISH may significantly increase the signal and achieve specific detection. Moreover, nuclampFISH may maintain the exponential amplification capacity of clampFISH. These methods may detect multiple nuclear RNAs, such as the long non-coding RNA (lncRNA) NEAT1. However, the total fluorescence produced for amplification strategies targeting NEAT1 may be lower than expected based on the amplification increase for cytoplasmic RNAs, suggesting that the labeling efficiency may be lower for nuclear RNAs.

As disclosed in Examples 4 and 5, cells were sorted based on the expression level of transcriptional bursts. The accuracy of the separation was demonstrated by both RT-qPCR and imaging. After sorting and collecting nuclei based on expression levels, crosslinking is reversed and a chromatin accessibility assay is performed for the gene of interest. Transcriptionally active cells were observed to have more open levels of chromatin than the transcription inactive cells. This provides direct evidence that open chromatin regions enable the binding of transcription factors and other regulatory elements to drive the transcription of a given gene. In the stochastic bursting model, a simplistic model fits kinetic information in a two-state system where the promoter alternates between an ON and OFF position [25]. This model has been expanded to include a third, ‘refractory’ promoter state where the promoter is bound but resistant to activation (i.e., no burst). The data shows that the cells exhibiting no bursting have significantly less “open chromatin” than the bursting cells, with levels comparable to that of negative control, HPRT1. This supports the two-state model.

In some embodiments, platforms disclosed herein combine single-cell analysis with bulk-cell sensitivity to move beyond an RNA-centric view of transcription and include the interacting factors of chromatin and protein in the analysis. In some embodiments, the sorting and crosslinking reversal steps enable downstream analysis such as chromatin capture, mass spectrometry (MS), and biochemical assays such as western blot and immunoprecipitation.

In some embodiments, methods for specific detection of nuclear RNA and transcription sites are disclosed herein. Such methods broadens the application of clampFISH for transcription sites by amplifying specific FISH signals, thus enabling the separation of the cells for downstream analysis, including chromatin analysis, proteomics, and transcriptional profiling. Example embodiments of the invention are useful for genome-wide assays. Example embodiments of the invention are useful for proteomic assays.

In some embodiments, methods of the present disclosure are used in cell analysis (e.g., rare cell analysis, analyzing transcripts in cancer cells such as melanoma cells). For example, methods of the present disclosure may be used to identify subpopulations of cancer cells that survive chemotherapy and may exhibit altered gene expression profiles indicative of drug resistance. In some embodiments, methods of the present disclosure are used in rare cell analysis (e.g., cells that make up less than about 1% of the total cell population, cells that remain after chemotherapy such as resistant subpopulations or cancer stem cells).

In some embodiments, nuclampFISH is established, to target nuclear RNA and transcription sites with a reversible crosslinker. An amplification strategy is used to sort cells based on transcriptional activity. A chromatin accessibility assay is performed to understand the differences between transcriptionally active and inactive cells.

### EXAMPLES

### Example 1

### ClampFISH and HCR Probes are Inaccessible to Transcription Sites.

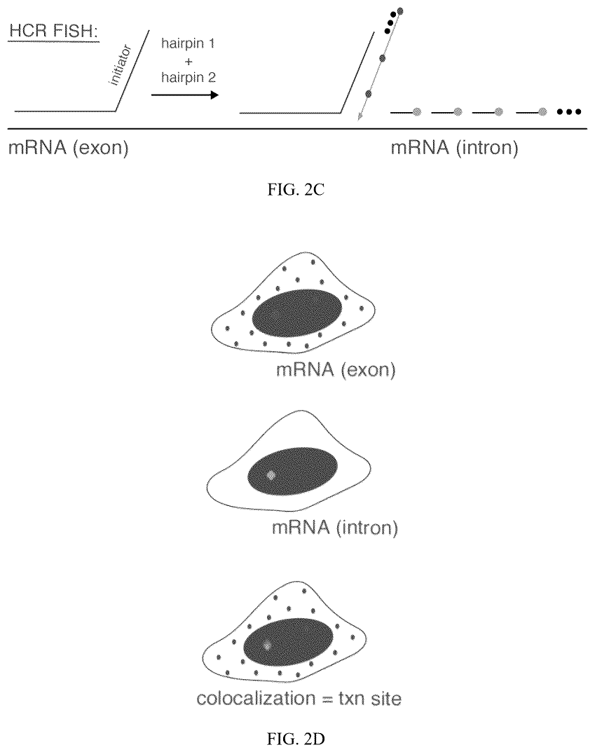

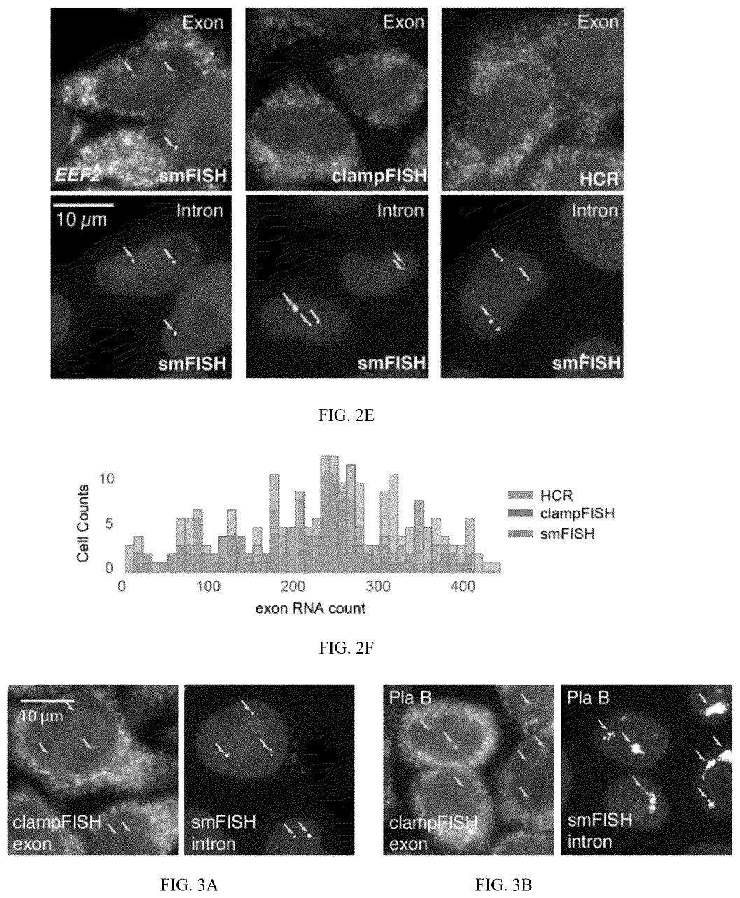

Previous reports have suggested that nuclear accessibility does not influence smFISH probes permeabilization (FIG. 2A), but it presents a challenge for FISH-based amplification, such as clampFISH and Hybridization chain reaction (HCR) (FIGS. 2B-C) [14]. Transcription sites reside in the nucleus and may be detected by the colocalization of exon and intron FISH probes for the same transcript (FIG. 2D). To assess the labeling efficiency of molecular scaffold-based amplification strategies for detecting transcription sites, EEF2 mRNA was labeled with smFISH, clampFISH, and HCR (FIG. 2E). This target has an average of 1.5 transcription sites in most HeLa cells, as detected by the colocalization of smFISH intron and exon probes in the nucleus (FIG. 2E). ClampFISH and HCR probes were designed to target the EEF2 exon sequence. They were counterstained by the smFISH probes that targeted the EEF2 intron. ClampFISH and HCR can reliably detect exon mRNA in cytoplasm, but the signal from the transcription site was lost or not specific. The nuclear exon spots overlaid well with nuclear intron spots (arrows), indicating that smFISH can detect transcription sites specifically (FIG. 2E). The distribution of exon FISH spots in clampFISH, HCR, and smFISH overlaps and the median number of the RNA spots counts in these three conditions are consistent: HCR: 250±4.2, clampFISH: 236±8.5, smFISH: 235±7.4 (FIG. 2F).

### Example 2

### Nuclear Isolation and Method Optimization Enable Exponential Amplification of Nuclear RNA.

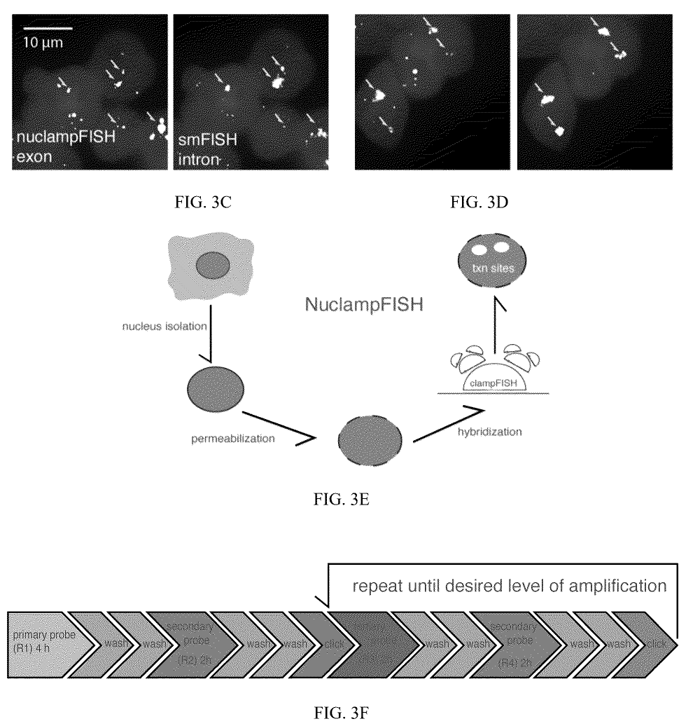

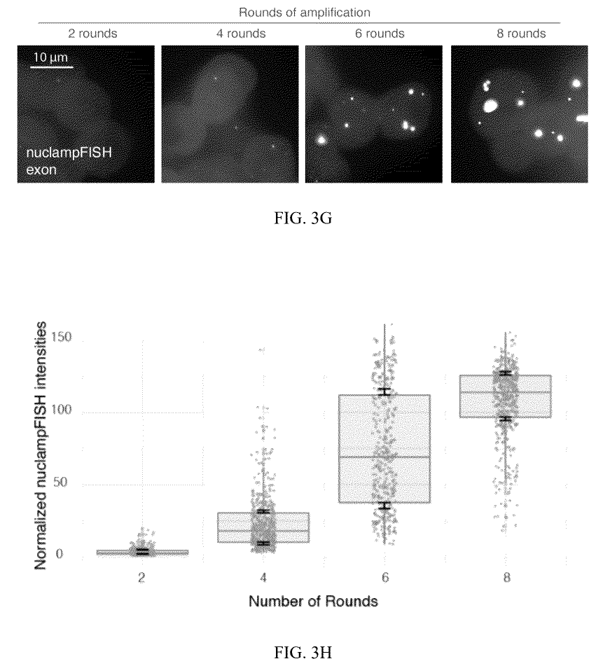

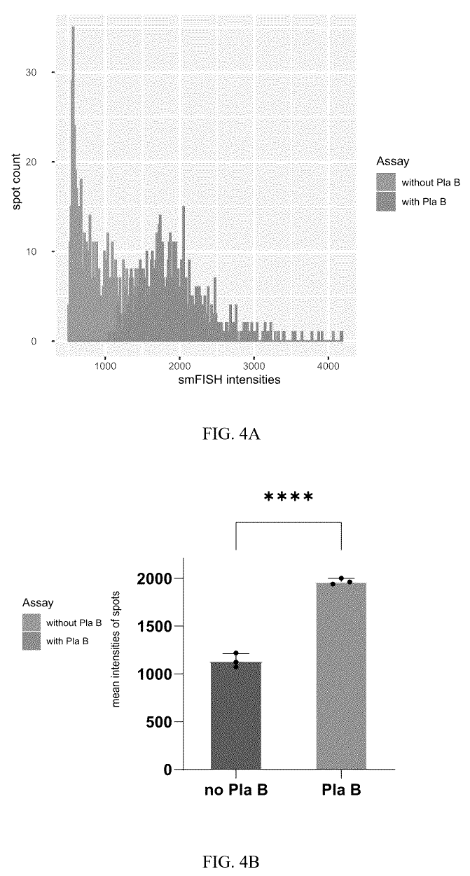

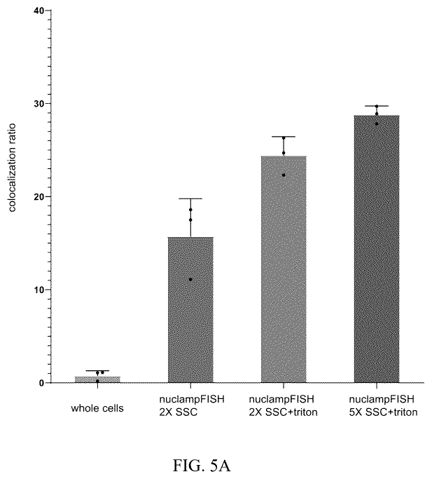

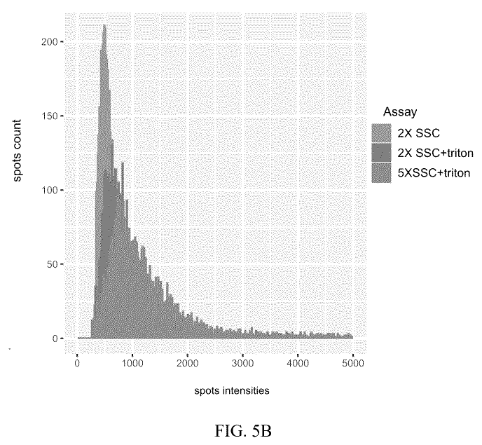

One hypothesis is that FISH amplification strategies are not capable of probing transcription sites because the probe binding is inefficient, and a larger transcriptional burst would not have this problem. To test whether increasing the number of nascent transcripts for a target transcript would enhance the signals to a detectable level, cells were treated with Pladienolide B (Pla B) [16]. This splicing inhibitor induces the transcription of EEF2 by blocking the assembling process of spliceosome [17]. ClampFISH probes were applied to target EEF2 exon using the original clampFISH protocol with and without the presence of Pla B. Even after Pla B treatment, clampFISH still exhibited a dim signal even though the smFISH signal had a 1.73-fold increase after Pla B treatment (FIGS. 3A-B, 4A-B) and minimal 0.78%+0.53% colocalization ratio with the intron smFISH probes (FIG. 5A-B).

The molecular scaffold size of the clampFISH probe may prevent itself from diffusing through the cytoplasm to access the target in the nucleus. The cell membrane and cytoplasm are removed, and then the clampFISH probes are applied to target EEF2 exons with two rounds of amplification (i.e., primary probe and secondary probe). After nuclear isolation, the signal intensities of transcription sites inside the cell nucleus and colocalization ratio with smFISH intron probes were improved significantly (FIGS. 3C, 5A-B) to 15.7%+4.1%. Next, the inclusion of surfactant and salt were investigated to improve probe binding. Triton™ X-100 enhances cell permeability, and salt drives probe hybridization with the target mRNA. After adding Triton™ X-100, nucleus clampFISH signal intensities and colocalization of clampFISH probes with smFISH intron probes increased to 24.4%+2.1% (FIGS. 3D, 5A-B). These values further increased by increasing the salt concentration from 2×SSC to 5×SSC (FIGS. 3D, 5A-B). The same result was observed for another nuclear RNA target, NEAT1, a long non-coding RNA (FIGS. 6A-B), CPSI intron (FIG. 7), and TMSF1 exons (FIG. 8).

Next, a method of probe delivery that would specifically and exponentially amplify the nuclear signal was tested. The same primary probes for the EEF2 exon were used and the signal was amplified using fluorescent secondary and tertiary probes with a click reaction performed (FIG. 3F). The nuclampFISH protocol was performed with varying stopping points: two rounds (primary and secondary), four rounds (primary, secondary, tertiary, and secondary again), six rounds, and eight rounds. Bright, amplified spots were observed with increasing fluorescent signal as amplification rounds increased (FIGS. 3G-H). Furthermore, an exponential rate of amplification was observed, 1.74-fold per round, indicating that the amplifiers' binding efficiency is 87.1% of the theoretical doubling intensity per round. This rate did not slow down even when eight rounds of amplification were reached, indicating that an even brighter signal could be achievable with additional amplification.

### Example 3

NuclampFISH is Compatible with Intron Targeting and Reversible Crosslinking.

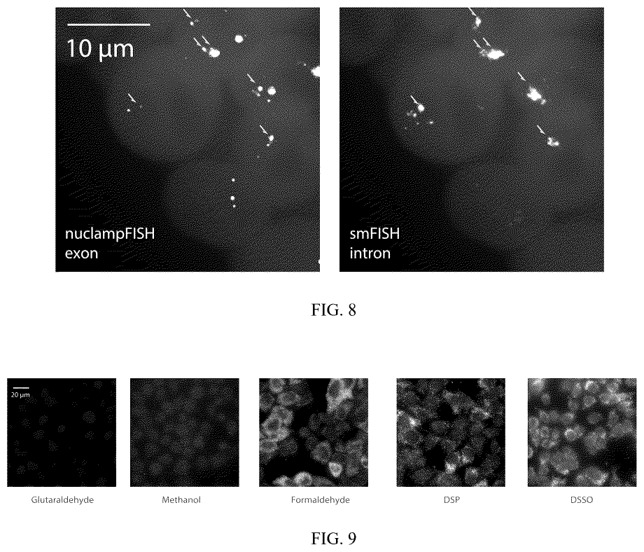

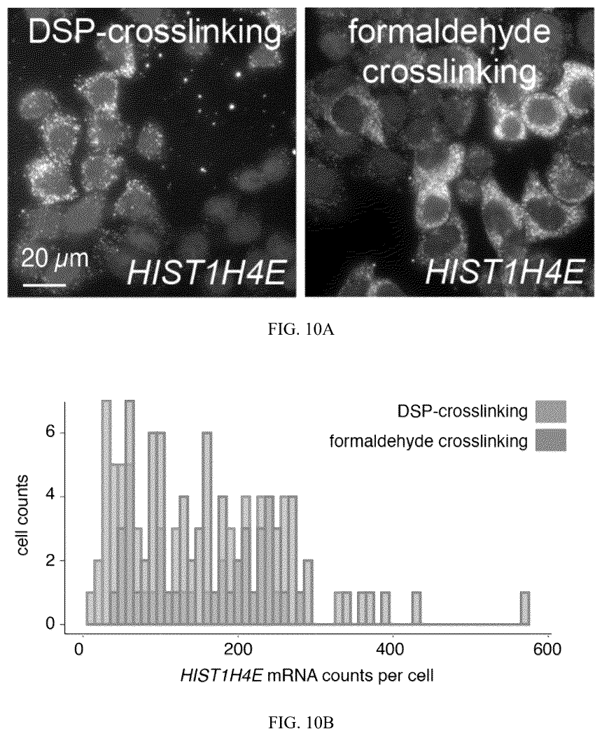

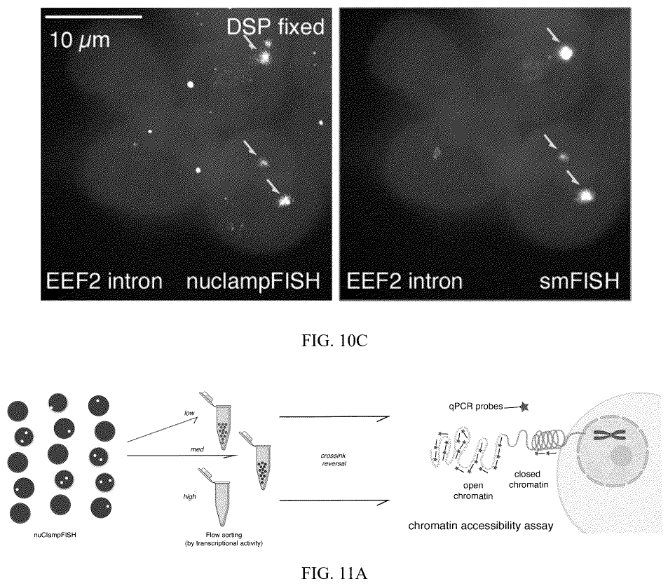

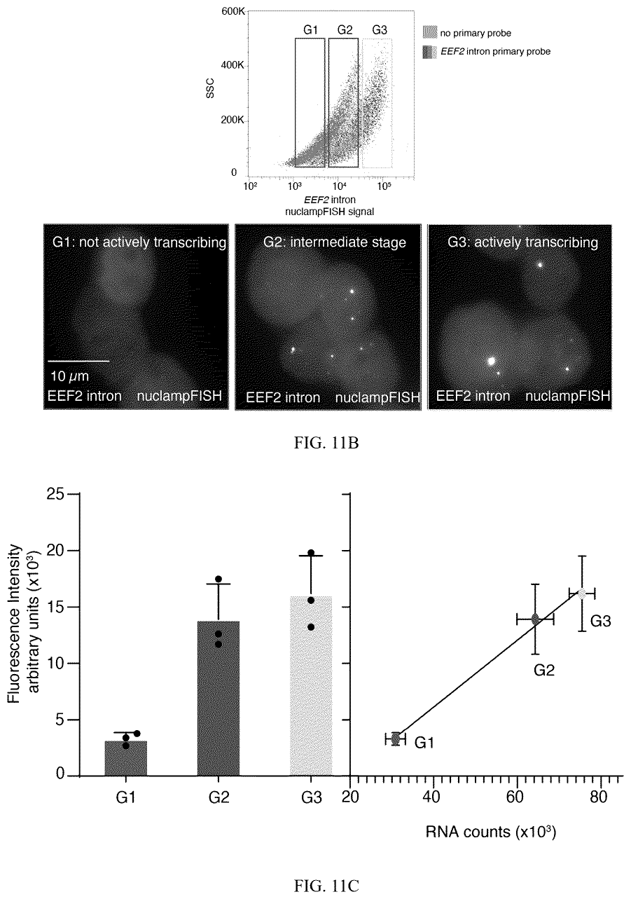

FISH-based methods require fixation and permeabilization before probe hybridization, including those with molecular scaffolding for fluorescence amplification. This fixation almost universally uses formaldehyde. Formaldehyde crosslinking is conventionally used for FISH-based assays [18,19]. However, it can interfere with downstream analysis, such as mass spectrometry, biochemical assays, and chromatin profiling [20,21]. To overcome the challenge of formaldehyde, the compatibility of several alternative crosslinkers with smFISH and clampFISH was tested, including glutaraldehyde, methanol, disuccinimidyl sulfoxide (DSSO), and dithiobis (succinimidyl propionate) (DSP). DSSO and DSP were found to have comparable performance with formaldehyde crosslinking (FIG. 9). DSP crosslinking was chosen because of its chemical reversibility, and DSSO is reversible by mass spectrometry. DSP could be applied to a broader variety of downstream applications. DSP is a crosslinker that can be easily reversed by 10-50 mM dithiothreitol (DTT) or tris(2-carboxyethyl) phosphine (TCEP) at pH 8.5 [22]. The performance of formaldehyde and DSP fixation was compared by targeting the HIST1H4E mRNAs using clampFISH exon probes (FIG. 10A). The RNA count distribution of DSP crosslinking can be overlaid well with formaldehyde crosslinking (FIG. 10B). The nuclampFISH method was applied to target transcription sites using DSP crosslinking with probes complementary to the EEF2 intron mRNA sequence. The nuclampFISH spots colocalized with smFISH spots at transcription sites, indicating that the protocol is compatible with DSP for targeting nuclear RNA while maintaining specificity (FIG. 10C).

### Example 4

### NuclampFISH Enables Cell Sorting Based on Nuclear RNA Expression.

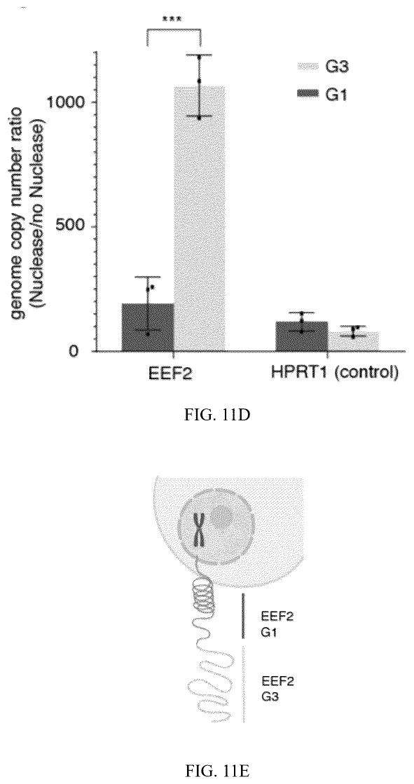

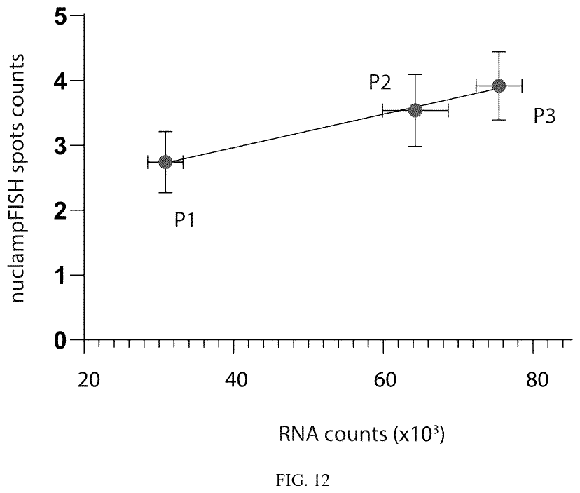

Cells may be sorted to isolate specific populations for further analysis, research, or therapeutic applications based on characteristics such as size, surface markers, and gene expression. One primary application of nuclampFISH is enablement of sorting cells based on nuclear RNA expression by detection of the active transcription sites of a single mRNA. NuclampFISH is used to sort cells based on the expression of actively transcribing EEF2 (i.e., clampFISH primary probes targeting the EEF2 intron). Compared to the negative control, which included each round of amplifier probe, the positive group, which was treated with four rounds of amplification, showed almost a complete decade shift (FIG. 11B). Three gates (G1, G2, G3 with increased nuclampFISH signal) were set to sort cells based on the expression level of transcription sites (FIG. 11B). After collecting the cells from each gate, the cells were imaged using fluorescence microscopy (FIG. 11B). The nuclampFISH signal on transcription sites, as shown by microscopy, corresponded to the increasing fluorescent intensity observed by flow cytometry (FIG. 11B-C). RNA is extracted from the sorted cells and RT-qPCR was conducted to quantify the levels of EEF2 intron from each gated population. The RNA counts correlated significantly with nuclampFISH pixel intensities acquired by microscopy after sorting (r=0.93; FIG. 11C) and nuclampFISH spots quantity (FIG. 12).

### Example 5

### NuclampFISH Enables Cell Sorting and Crosslink Reversal for Chromatin Accessibility Analysis.

Chromatin accessibility assays such as DNA sequencing-based methods facilitate characterizing active regulatory elements during active transcription [23,24]. However, these are typically bulk assays that combine heterogeneous cell populations for analysis rather than single-cell assays. Single-cell assays such as smFISH can analyze gene-specific, nascent RNA; however, these assays do not inform DNA accessibility. The single-cell and gene-specific benefits of FISH-based assays were sought to be combined with the sensitivity of bulk assays to detect chromatin structure and explore chromatin conformation during active transcription of a given gene.

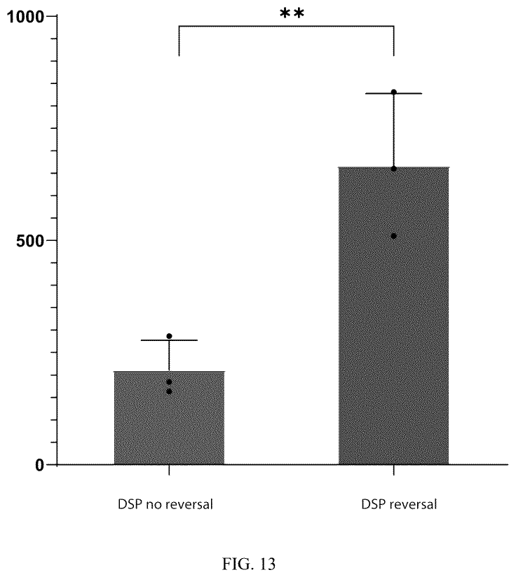

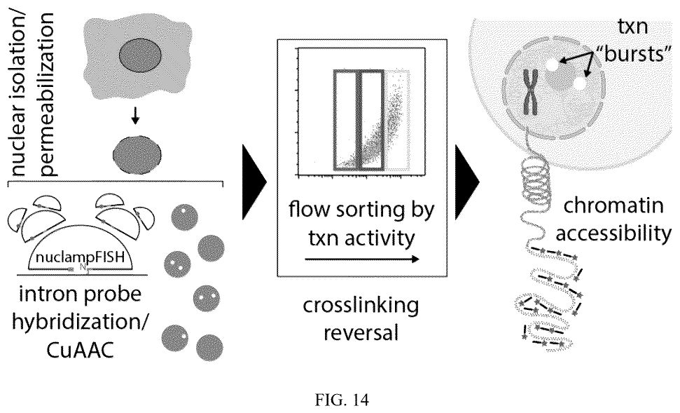

As a first step, an assay was performed to determine whether the chromatin accessibility assay may be compatible with DSP crosslinked cells for which the crosslinking was reversed. Chromatin accessibility assays report on the “openness” of chromatin based on the effectiveness of qPCR in a region of interest. Crosslinking the nucleic acids and proteins surrounding the region of interest can lead to misinterpretation of the results as closed when it could be a crosslinking artifact. As a proof-of-concept, cells were crosslinked with DSP, and the EEF2 chromatin accessibility of the DSP reversal group was compared with the no DSP reversal group; the reversal group exhibited a 3.16-fold increase in signal (FIG. 13), indicating that crosslink reversal is compatible with the chromatin accessibility assay.

Next, HeLa cells were crosslinked with DSP, EEF2 introns were labeled using nuclampFISH with four rounds of amplification, and the sorted nucleus were collected from the G1 and G3 groups. Crosslinking was reversed from the G1 and G3 groups, followed by chromatin accessibility analysis for the genomic region harboring the EEF2 intron. The G3 group (i.e., the group with a higher level of “burstiness” for EEF2 mRNA) had a 5.59-fold increase in accessibility compared to the G1 group (i.e., the group with limited “burstiness” for EEF2 mRNA; p<0.0001; FIG. 11D). As a critical control, a housekeeping gene, HPRT1, was selected and the chromatin accessibility assay was performed on the G1 and G3 groups selected for EEF2. HPRT1 did not show a significant difference in chromatin accessibility (FIG. 11D). These results demonstrate that “bursty” transcription corresponds with more accessible chromatin at the gene of interest (FIG. 11E).

### Experimental Methods for Examples 1-5

### Tissue Culture Conditions

HeLa cells were handled according to the manufacturer's protocol and maintained in high-glucose DMEM with GlutaMax (FisherSci cat #10566016), 10% FBS, and 1% Pen-Strep (FisherSci cat #BW17-602E). Cell lines tested PCR-negative for mycoplasma contamination (ATCC cat #30-1012K).

### Drug Treatment for Splicing Inhibition

Cells were treated with 1 μM Pladienolide B (Tocris Biosciences #6070500U) for 4 h to achieve splicing inhibition as described by previous work [26].

smFISH

The smFISH detection was performed as previously described [19]. Sequences for smFISH probes can be found in Tables 1-3.

TABLE 1
EEF2 exon smFISH probes. TEG: Tetraethylene
Glycol. mdC: 5-methyl-deoxycytidine.

| EEF2 exon | | SEQ |
|---|---|---|
| smFISH | | ID |
| probes | | NO: |
| EEF2_exon1 | cccaggtagaaccgaaagaa | 1 |
| | mdC(TEG-Amino) | |
| EEF2_exon4 | gtatcagtgaagcgtgtctc | 2 |
| | mdC(TEG-Amino) | |
| EEF2_exon6 | gctcgtagaagagggagatg | 3 |
| | mdC(TEG-Amino) | |
| EEF2_exon8 | tccgaggagaagtcgacatg | 4 |
| | mdC(TEG-Amino) | |
| EEF2_exon9 | cttgttcatcatcagcacag | 5 |
| | mdC(TEG-Amino) | |
| EEF2_exon10 | acgatgcgctggaaagtctg | 6 |
| | mdC(TEG-Amino) | |
| EEF2_exon12 | cgaggacaggatcgatcatg | 7 |
| | mdC(TEG-Amino) | |
| EEF2_exon15 | ttcatcatgtcctctacttt | 8 |
| | mdC(TEG-Amino) | |
| EEF2_exon16 | tggctgacttgctgaacttg | 9 |
| | mdC(TEG-Amino) | |
| EEF2_exon19 | tgatgtccagtttctctatc | 10 |
| | mdC(TEG-Amino) | |
| EEF2_exon21 | tttggggtcacagcttttaa | 11 |
| | mdC(TEG-Amino) | |
| EEF2_exon25 | gcccatcatcaagattgttc | 12 |
| | mdC(TEG-Amino) | |
| EEF2_exon26 | aatgttcccacaaggcacat | 13 |
| | mdC(TEG-Amino) | |
| EEF2_exon27 | cgtgctcgaaggtggtgatg | 14 |
| | mdC(TEG-Amino) | |
| EEF2_exon29 | actctgacaacagggctgac | 15 |
| | mdC(TEG-Amino) | |
| EEF2_exon30 | cgatgatgcactgcaccatg | 16 |
| | mdC(TEG-Amino) | |
| EEF2_exon31 | gagacgaccgggtcagattt | 17 |
| | mdC(TEG-Amino) | |
| EEF2_exon33 | ttgttgggggacttggagag | 18 |
| | mdC(TEG-Amino) | |
| EEF2_exon34 | caaagcaccagatcttgcgg | 19 |
| | mdC(TEG-Amino) | |
| EEF2_exon35 | tgatgtcggtgaggatgttg | 20 |
| | mdC(TEG-Amino) | |
| EEF2_exon36 | cttgatctcgttgaggtact | 21 |
| | mdC(TEG-Amino) | |
| EEF2_exon37 | tgacgtcgtggacgtcgaag | 22 |
| | mdC(TEG-Amino) | |
| EEF2_exon38 | gtcagcacactggcatagag | 23 |
| | mdC(TEG-Amino) | |
| EEF2_exon39 | aaggtagatgggctccatga | 24 |
| | mdC(TEG-Amino) | |
| EEF2_exon40 | tgttcaaaaccccgtagatg | 25 |
| | mdC(TEG-Amino) | |
| EEF2_exon42 | cctacaatttgtccaggaag | 26 |
| | mdC(TEG-Amino) | |
| EEF2_exon43 | tccaggtgtcgtctgagaat | 27 |
| | mdC(TEG-Amino) | |
| EEF2_exon45 | ccattaagtcectactaaga | 28 |
| | mdC(TEG-Amino) | |
| EEF2_exon46 | ttctggggcaaaagccactg | 29 |
| | mdC(TEG-Amino) | |
| EEF2_exon48 | caatggcactagtacagctg | 30 |
| | mdC(TEG-Amino) | |

Note:
this is a subset of the full EEF2 exon smFISH set that does not interfere with EEF2 exon clampFISH probe binding

TABLE 2
EEF2 intron smFISH probes

| EEF2 intron | | |
|---|---|---|
| smFISH | | SEQ |
| probes (for | | ID |
| colocalization) | | NO: |
| EEF2_intron1 | cctcggaaacggagaaaggg | 31 |
| | mdC(TEG-Amino) | |
| EEF2_intron4 | ttggcaaacgggatcagaca | 32 |
| | mdC(TEG-Amino) | |
| EEF2_intron5 | atttggggccaaacacgtaa | 33 |
| | mdC(TEG-Amino) | |
| EEF2_intron6 | ttccttgataccaggtacaa | 34 |
| | mdC(TEG-Amino) | |
| EEF2_intron7 | ccaaggaaccaccgttaata | 35 |
| | mdC(TEG-Amino) | |
| EEF2_intron8 | agcgttcagtccaaacgaac | 36 |
| | mdC(TEG-Amino) | |
| EEF2_intron9 | tcgtgatttccaggaacaca | 37 |
| | mdC(TEG-Amino) | |
| EEF2_intron12 | ctcaagaaaaccttccagct | 38 |
| | mdC(TEG-Amino) | |
| EEF2_intron14 | agatattgtaggagtggggg | 39 |
| | mdC(TEG-Amino) | |
| EEF2_intron15 | gaacatcatccagtgcaaca | 40 |
| | mdC(TEG-Amino) | |
| EEF2_intron17 | ctaaaagacccagcaggacg | 41 |
| | mdC(TEG-Amino) | |
| EEF2_intron18 | agattcatcacacctgactc | 42 |
| | mdC(TEG-Amino) | |
| EEF2_intron19 | aaaaacacacttccagtccc | 43 |
| | mdC(TEG-Amino) | |
| EEF2_intron21 | tcttcagtagacatctggtc | 44 |
| | mdC(TEG-Amino) | |
| EEF2_intron22 | aaacgagaagcagccgtgag | 45 |
| | mdC(TEG-Amino) | |
| EEF2_intron24 | gcagtccagatcttaagaga | 46 |
| | mdC(TEG-Amino) | |
| EEF2_intron25 | gccaaagccacaagttattc | 47 |
| | mdC(TEG-Amino) | |
| EEF2_intron28 | gaagccaaggaagccaaagt | 48 |
| | mdC(TEG-Amino) | |
| EEF2_intron29 | gacagagaaaacccgcaagc | 49 |
| | mdC(TEG-Amino) | |
| EEF2_intron30 | tctatgctccttacttctag | 50 |
| | mdC(TEG-Amino) | |
| EEF2_intron31 | acaaaagttgtggctggcac | 51 |
| | mdC(TEG-Amino) | |
| EEF2_intron32 | ccacctggaagaagcagaag | 52 |
| | mdC(TEG-Amino) | |
| EEF2_intron35 | gccagaacaagatttcaggg | 53 |
| | mdC(TEG-Amino) | |
| EEF2_intron36 | tttaaagcagagggcaggtg | 54 |
| | mdC(TEG-Amino) | |
| EEF2_intron38 | agcatttgtgttgtttgtgg | 55 |
| | mdC(TEG-Amino) | |
| EEF2_intron41 | ctcactccagacaaggacaa | 56 |
| | mdC(TEG-Amino) | |
| EEF2_intron42 | gccacgtcaatcagaacgaa | 57 |
| | mdC(TEG-Amino) | |
| EEF2_intron45 | tgcctcacctgcaaaaagag | 58 |
| | mdC(TEG-Amino) | |
| EEF2_intron46 | acctgccagaagggaaagaa | 59 |
| | mdC(TEG-Amino) | |
| EEF2_intron48 | attcagtgatttagggggtc | 60 |
| | mdC(TEG-Amino) | |

Note:
this is a subset of the full EEF2 intron smFISH set that does not interfere with EEF2 intron clampFISH probe binding

TABLE 3
TMSF1 intron smFISH probes

| TMSF1 intron | | |
|---|---|---|
| smFISH | | SEQ |
| probes (for | | ID |
| colocalization) | | NO: |
| TMSF1_intron1 | tcctacctatagagtttagc | 61 |
| | mdC(TEG-Amino) | |
| TMSF1_intron2 | actattagcttcagacaccg | 62 |
| | mdC(TEG-Amino) | |
| TMSF1_intron3 | ctgtggttgaagctaatgct | 63 |
| | mdC(TEG-Amino) | |
| TMSF1_intron4 | agatttgccatgccataatc | 64 |
| | mdC(TEG-Amino) | |
| TMSF1_intron5 | gaccccagataacaaactct | 65 |
| | mdC(TEG-Amino) | |
| TMSF1_intron6 | cacagctacatcttaggagt | 66 |
| | mdC(TEG-Amino) | |
| TMSF1_intron7 | tttgatagagctgccactat | 67 |
| | mdC(TEG-Amino) | |
| TMSF1_intron8 | tgcctgagtcagtgagaata | 68 |
| | mdC(TEG-Amino) | |
| TMSF1_intron9 | tcaggcaattgttctggtat | 69 |
| | mdC(TEG-Amino) | |
| TMSF1_intron10 | attcatcaccacatacatcc | 70 |
| | mdC(TEG-Amino) | |
| TMSF1_intron11 | gtacatccagtatgttctga | 71 |
| | mdC(TEG-Amino) | |
| TMSF1_intron12 | tgcattctggttgcataagt | 72 |
| | mdC(TEG-Amino) | |
| TMSF1_intron13 | cccatattgaaatccttgga | 73 |
| | mdC(TEG-Amino) | |
| TMSF1_intron14 | gtggggttttaatcttaggg | 74 |
| | mdC(TEG-Amino) | |
| TMSF1_intron15 | acttccttccacaacagaac | 75 |
| | mdC(TEG-Amino) | |
| TMSF1_intron16 | tcagcaaaccatactgaggt | 76 |
| | mdC(TEG-Amino) | |
| TMSF1_intron17 | acacacggtcatacttgagg | 77 |
| | mdC(TEG-Amino) | |
| TMSF1_intron18 | tggtatattaacaccagggc | 78 |
| | mdC(TEG-Amino) | |
| TMSF1_intron19 | gaagcttgttactgacagct | 79 |
| | mdC(TEG-Amino) | |
| TMSF1_intron20 | cagcatgttcttgtgtacat | 80 |
| | mdC(TEG-Amino) | |
| TMSF1_intron21 | ttttgcacagagcgactcaa | 81 |
| | mdC(TEG-Amino) | |
| TMSF1_intron22 | ttcctggttttctttcacaa | 82 |
| | mdC(TEG-Amino) | |
| TMSF1_intron23 | ccctctcaaagataggatca | 83 |
| | mdC(TEG-Amino) | |
| TMSF1_intron24 | gctatacttgcctgataagg | 84 |
| | mdC(TEG-Amino) | |
| TMSF1_intron25 | cacttactttgaactctgcc | 85 |
| | mdC(TEG-Amino) | |
| TMSF1_intron26 | tttggtcattggctctttat | 86 |
| | mdC(TEG-Amino) | |
| TMSF1_intron27 | ctgggaaacctggtgtattt | 87 |
| | mdC(TEG-Amino) | |
| TMSF1_intron28 | gcaggatactgatattgggt | 88 |
| | mdC(TEG-Amino) | |
| TMSF1_intron29 | aaacaaacccagatcccttg | 89 |
| | mdC(TEG-Amino) | |
| TMSF1_intron30 | tcttagcttcagcaaagctt | 90 |
| | mdC(TEG-Amino) | |

Note:
this is a subset of the full TMSF1 intron smFISH set that does not interfere with TMSF1 intron clampFISH probe binding

### HCR FISH

HCR FISH was conducted using the HCR™ RNA-FISH Bundle from Molecular Instruments. The manufacturer's protocol was followed [11, 27].

### NuclampFISH Protocol

### Nucleus Extraction

Minute™ Single Nucleus Isolation Kit for Tissues/Cells (Invent Biotechnologies cat #SN-047) was used to extract nuclei from whole cells. 5×10⁶-10⁷ cells were collected and washed by pre-cold 1×PBS once. 200 μL lysis buffer was added to the tube, and the cells were ground with the pestle provided 20-30 times. Another 400 μL lysis buffer was added to the tube and incubated at 4° C. for 10 min. After incubation, all cell lysate was transferred into a filter in a collection tube. The nuclei pellet was obtained by centrifuge at 600×g for 5 min. The supernatant was removed carefully and discarded. The pellet was resuspended in 0.8 mL cold washing buffer by pipetting up and down 20-30 times. The washed nuclei were centrifuged at 500×g for 5 min. The supernatant was removed and discarded. The nuclei were ready to proceed into the fixation step.

### Formaldehyde and DSP Fixation

After cell nuclei were isolated and washed, 3.7% formaldehyde (1×PBS and diluted in Nuclease-Free water) and 500 μM DSP (ThermoFisher #PIA35393, 1 mg stock dissolved in 50 μL DMSO and then diluted in 1×PBS) were directly added to the pellet to fix nuclei, the incubation time was 10 min. For 5×10⁶-10⁷ cells, 5 mL 3.7% formaldehyde and DSP were added. After fixation, the nuclei were incubated with 1×PBS/0.1% Triton™ X-100 for 5 min twice to improve permeabilization. After this, the nuclei were incubated with clampFISH wash buffer/0.25% Triton™ X-100 for 20 min to achieve further permeabilization. The nuclei were ready to do clampFISH treatment.

clampFISH

The clampFISH procedure was conducted as in previous work with several modifications to increase nuclear RNA FISH signal.

Sequences for clampFISH probes are found in Tables 4-8.

Cells were cultured on glass coverslips until they reached approximately 70% confluence. The cells were washed twice with PBS and then fixed in 4% formaldehyde in PBS at room temperature for 10 minutes. After aspirating the formaldehyde, cells were rinsed twice with PBS and stored them in 70% ethanol at 4° C. For hybridization, cells were incubated for at least 4 hours at 37° C. in a buffer containing 10% dextran sulfate, 5×SSC, 0.25% Triton-X-100 and 20% formamide, along with 0.5 μL of the primary clampFISH probe. Probes were designed according to Rouhanifard et al. [14].

Next, two 30-minute washes were performed at 37° C. in wash buffer (2×SSC, 10% formamide). Cells were incubated for at least 2 hours at 37° C. in a hybridization buffer with 1 μL of the secondary clampFISH probe, followed by the same wash procedure. After the second wash, the click chemistry reaction was initiated by adding a solution of 75 μM CuSO₄ premixed with 150 μM BTTAA ligand (Jena Biosciences) and 2.5 mM freshly prepared sodium ascorbate (Sigma) in 2×SSC. The samples were incubated for 30 minutes at 37° C. and briefly rinsed with wash buffer.

Amplification cycles were continued by alternating between secondary and tertiary clampFISH probes, followed by the respective wash steps until the desired signal amplification was achieved. After the final wash, the cells were rinsed once with 2×SSC containing DAPI and once with antifade buffer (10 mM Tris, pH 8.0, 2×SSC, 1% w/v glucose). The samples were then mounted for imaging in an antifade buffer containing catalase (Sigma) and glucose oxidase (Sigma) to prevent photobleaching.

For suspension cells, 0.25% Triton X-100 was added to the wash buffer; the cells were stored in 2×SSC/0.25% triton after clampFISH with desired rounds ready for flow cytometry sorting.

TABLE 4
Sequences of primary EEF2 exon clampFISH probes,
secondary, and tertiary clampFISH probes.

| Primary | | |
|---|---|---|
| EEF2 exon | | SEQ |
| clampFISH | | ID |
| probes: | | NO: |
| leftarm_ | | |
| sequence | | |
| EEF2_exon_ | /5Hexynyl/tgttgcggatgttggACATCATAGT | 91 |
| P1 | | |
| EEF2_exon_ | /5Hexynyl/tgatgaggaagccggACATCATAGT | 92 |
| P2 | | |
| EEF2_exon_ | /5Hexynyl/gatgacgttcacgttACATCATAGT | 93 |
| P3 | | |
| EEF2_exon_ | /5Hexynyl/caaactgcttcagggACATCATAGT | 94 |
| P4 | | |
| EEF2_exon_ | /5Hexynyl/ttgaagatggggtccACATCATAGT | 95 |
| P5 | | |
| EEF2_exon_ | /5Hexynyl/tctgcaacaaggcgtACATCATAGT | 96 |
| P6 | | |
| EEF2_exon_ | /5Hexynyl/accgacctttgtcggACATCATAGT | 97 |
| P7 | | |
| EEF2_exon_ | /5Hexynyl/gactcgttgacgggcACATCATAGT | 98 |
| P8 | | |
| EEF2_exon_ | /5Hexynyl/gttgagtgatggcacACATCATAGT | 99 |
| P9 | | |
| EEF2_exon_ | /5Hexynyl/aaatcatctacccgcACATCATAGT | 100 |
| P10 | | |
| backbone_ | | |
| sequence | | |
| (P9 | | |
| backbone) | | |
| EEF2_exon_ | /5Phos/atattcgcagtgcattactggtAGTGGT | 101 |
| P1-P10 | AGGTAGACATTCatagtacagatgcattactggtA | |
| | GTGGTAGGTAGACATTCctgagtgttg | |
| rightarm_ | | |
| sequence | | |
| EEF2_exon_ | /5Phos/aagtgactgtgggcgatgacagaca/ | 102 |
| P1 | 3AzideN/ | |
| EEF2_exon_ | /5Phos/aagtgactgtgggagtcaatgaggt/ | 103 |
| P2 | 3AzideN/ | |
| EEF2_exon_ | /5Phos/aagtgactgtgccgtaggtggagat/ | 104 |
| P3 | 3AzideN/ | |
| EEF2_exon_ | /5Phos/aagtgactgtccacatacatctcgg/ | 105 |
| P4 | 3AzideN/ | |
| EEF2_exon_ | /5Phos/aagtgactgtatcgcatcaaacacc/ | 106 |
| P5 | 3AzideN/ | |
| EEF2_exon_ | /5Phos/aagtgactgtggtggatggtgatca/ | 107 |
| P6 | 3AzideN/ | |
| EEF2_exon_ | /5Phos/aagtgactgtgtccaaaggcgtaga/ | 108 |
| P7 | 3AzideN/ | |
| EEF2_exon_ | /5Phos/aagtgactgtgcggtgaagccaaag/ | 109 |
| P8 | 3AzideN/ | |
| EEF2_exon_ | /5Phos/aagtgactgtgcatcaagtgttatg/ | 110 |
| P9 | 3AzideN/ | |
| EEF2_exon_ | /5Phos/aagtgactgtcttcctgcctgctag/ | 111 |
| P10 | 3AzideN/ | |
| left_ | | |
| adapter | | |
| EEF2_exon_ | ctgcgaatatACTATGATGT | 112 |
| P1-P10 | | |
| right_ | | |
| adapter | | |
| EEF2_exon_ | acagtcacttcaacactcag | 113 |
| P1-P10 | | |
| Secondary | | |
| clampFISH | | |
| probes: | | |
| MM2_Cy5_ | /5Hexynyl/ACTaccagtaatgca/iCy5/TTTT | 114 |
| leftarm_ | AATATT | |
| sequence | | |
| MM2_ | /5Phos/caatgagttaCAGGAGTTGTGTTTGTGG | 115 |
| backbone | ACGAAGAGCACCatcatattaaggCAGGAGTTGTG | |
| | TTTGTGGACGAAGAGCACCataagaatca | |
| MM2_ | /5Phos/AAGTGACTGTGAATGTCTACCTACC/ | 116 |
| rightarm_ | 3AzideN/ | |
| sequence | | |
| MM2_left_ | TAACTCATTGAATATTAAAA | 117 |
| adapter | | |
| MM2_right_ | ACAGTCACTTTGATTCTTAT | 118 |
| adapter | | |
| Tertiary | | |
| clampFISH | | |
| probes: | | |
| P9_Cy5_ | /5Hexynyl/CAAACACAACTCCTG/iCy5/ | 119 |
| leftarm_ | ACATCATAGT | |
| sequence | | |
| P9_ | /5Phos/atattcgcagtgcattactggtAGTGGT | 101 |
| backbone | AGGTAGACATTCatagtacagatgcattactggtA | |
| | GTGGTAGGTAGACATTCctgagtgttg | |
| P9_ | /5Phos/AAGTGACTGTGGTGCTCTTCGTCCA/ | 120 |
| rightarm_ | 3AzideN/ | |
| sequence | | |
| P9_left_ | ctgcgaatatACTATGATGT | 112 |
| adapter | | |
| P9_right_ | acagtcacttcaacactcag | 113 |
| adapter | | |

5Hexynyl: 5′-hexynyl.
5Phos: 5′-phosphate.
3AzideN: 3′-azide.
Nucleotides that are underlined represent regions which are complementary to the left or right adapters, in an embodiment.
The italicized and bolded nucleotides are complementary to the left arm and right arm binding sequence, respectively, for subsequent amplification probe (e.g., secondary clampFISH probe, tertiary clampFISH probe), in an embodiment.

TABLE 5
Sequences of primary HIST1H4E clampFISH probes.

| Primary | | |
|---|---|---|
| HIST1H4E | | SEQ |
| clampFISH | | ID |
| probes: | | NO: |
| leftarm_ | | |
| sequence | | |
| HIST1H4E_ | /5hexynyl/GCCGCGACCAGACATACATCATAGT | 121 |
| P1 | | |
| HIST1H4E_ | /5hexynyl/CGCCTCCTTTACCCAACATCATAGT | 122 |
| P2 | | |
| HIST1H4E_ | /5hexynyl/TTATCTCGCAGGACCACATCATAGT | 123 |
| P3 | | |
| HIST1H4E_ | /5hexynyl/CCGGATGGCAGGCTTACATCATAGT | 124 |
| P4 | | |
| HIST1H4E_ | /5hexynyl/TGCGCTTGACACCCCACATCATAGT | 125 |
| P5 | | |
| HIST1H4E_ | /5hexynyl/CCGCGAGTCTCCTCGACATCATAGT | 126 |
| P6 | | |
| backbone_ | | |
| sequence | | |
| (P9 | | |
| backbone) | | |
| HIST1H4E_ | /5Phos/atattcgcagtgcattactggtAGTGGT | 101 |
| P1-P6 | AGGTAGACATTCatagtacagatgcattactggtA | |
| | GTGGTAGGTAGACATTCctgagtgttg | |
| rightarm_ | | |
| sequence | | |
| HIST1H4E_ | /5Phos/aagtgactgtTCCCTTTCCGCCTTT/ | 127 |
| P1 | 3AzideN/ | |
| HIST1H4E_ | /5Phos/aagtgactgtTACGGTGACGCTTAG/ | 128 |
| P2 | 3AzideN/ | |
| HIST1H4E_ | /5Phos/aagtgactgtGTAATGCCCTGGATG/ | 129 |
| P3 | 3AzideN/ | |
| HIST1H4E_ | /5Phos/aagtgactgtGCGACGAGCAAGGCG/ | 130 |
| P4 | 3AzideN/ | |
| HIST1H4E_ | /5Phos/aagtgactgtAGATGAGACCAGAAA/ | 131 |
| P5 | 3AzideN/ | |
| HIST1H4E_ | /5Phos/aagtgactgtAACACCTTCAGAACC/ | 132 |
| P6 | 3AzideN/ | |
| left_ | | |
| adapter | | |
| HIST1H4E_ | ctgcgaatatACTATGATGT | 112 |
| P1-P6 | | |
| right_ | | |
| adapter | | |
| HIST1H4E_ | acagtcacttcaacactcag | 113 |
| P1-P6 | | |

Nucleotides that are underlined represent regions which are complementary to the left or right adapters, in an embodiment. The italicized and bolded nucleotides are complementary to the left arm and right arm binding sequence respectively for subsequent amplification probe (e.g., secondary clampFISH probe, tertiary clampFISH probe), in an embodiment.

TABLE 6
Sequences of primary EEF2 intron
clampFISH probes.

| Primary | | |
|---|---|---|
| EEF2 intron | | SEQ |
| clampFISH | | ID |
| probes: | | NO: |
| leftarm_ | | |
| sequence | | |
| EEF2_intron_ | /5Hexynyl/tctcaataagagtctACATCATAGT | 133 |
| P1 | | |
| EEF2_intron_ | /5Hexynyl/cttcatcataaaatgACATCATAGT | 134 |
| P2 | | |
| EEF2_intron_ | /5Hexynyl/taagaaaccaggggaACATCATAGT | 135 |
| P3 | | |
| EEF2_intron_ | /5Hexynyl/gatgaaaacattccaACATCATAGT | 136 |
| P4 | | |
| EEF2_intron_ | /5Hexynyl/aagttagggtctccaACATCATAGT | 137 |
| P5 | | |
| EEF2_intron_ | /5Hexynyl/agaggaaaaagcccaACATCATAGT | 138 |
| P6 | | |
| EEF2_intron_ | /5Hexynyl/atgttaagtcccacaACATCATAGT | 139 |
| P7 | | |
| EEF2_intron_ | /5Hexynyl/taagaagctgagaccACATCATAGT | 140 |
| P8 | | |
| EEF2_intron_ | /5Hexynyl/aacgaggtctggagcACATCATAGT | 141 |
| P9 | | |
| EEF2_intron_ | /5Hexynyl/tcctagcaaggcggaACATCATAGT | 142 |
| P10 | | |
| backbone_ | | |
| sequence | | |
| (P9 | | |
| backbone) | | |
| EEF2_intron_ | /5Phos/atattcgcagtgcattactggtAGTGGT | 101 |
| P1-P10 | AGGTAGACATTCatagtacagatgcattactggtA | |
| | GTGGTAGGTAGACATTCctgagtgttg | |
| rightarm_ | | |
| sequence | | |
| EEF2_intron_ | /5Phos/aagtgactgtagcctacacctcgtg/ | 143 |
| P1 | 3AzideN/ | |
| EEF2_intron_ | /5Phos/aagtgactgttccccgagcaagaag/ | 144 |
| P2 | 3AzideN/ | |
| EEF2_intron_ | /5Phos/aagtgactgtcattctcccatgaat/ | 145 |
| P3 | 3AzideN/ | |
| EEF2_intron_ | /5Phos/aagtgactgtggctgggtcaagtcg/ | 146 |
| P4 | 3AzideN/ | |
| EEF2_intron_ | /5Phos/aagtgactgtaggccaagccaaatc/ | 147 |
| P5 | 3AzideN/ | |
| EEF2_intron_ | /5Phos/aagtgactgtacctcgtggtggagc/ | 148 |
| P6 | 3AzideN/ | |
| EEF2_intron_ | /5Phos/aagtgactgtcaaactcctgaagaa/ | 149 |
| P7 | 3AzideN/ | |
| EEF2_intron_ | /5Phos/aagtgactgtgaaacttgaggaact/ | 150 |
| P8 | 3AzideN/ | |
| EEF2_intron_ | /5Phos/aagtgactgtcattaacagggaaga/ | 151 |
| P9 | 3AzideN/ | |
| EEF2_intron_ | /5Phos/aagtgactgtatccttaaaagcttt/ | 152 |
| P10 | 3AzideN/ | |
| left_ | | |
| adapter | | |
| EEF2_intron_ | ctgcgaatatACTATGATGT | 112 |
| P1-P10 | | |
| right_ | | |
| adapter | | |
| EEF2_intron_ | acagtcacttcaacactcag | 113 |
| P1-P10 | | |

Nucleotides that are underlined represent regions which are complementary to the left or right adapters, in an embodiment. The italicized and bolded nucleotides are complementary to the left arm and right arm binding sequence respectively for subsequent amplification probe (e.g., secondary clampFISH probe, tertiary clampFISH probe), in an embodiment.

TABLE 7
Sequences of primary NEATI clampFISH probes.

| Primary | | |
|---|---|---|
| NEAT1 | | SEQ |
| clampFISH | | ID |
| probes: | | NO: |
| leftarm_ | | |
| sequence | | |
| NEAT1_P1 | /5hexynyl/TGAGCTAGCCACTTCACATCATAGT | 153 |
| NEAT1_P2 | /5hexynyl/CCCAGAGGTCAAGTTACATCATAGT | 154 |
| NEAT1_P3 | /5hexynyl/GGCTCGCTCAGCTATACATCATAGT | 155 |
| NEAT1_P4 | /5hexynyl/CTGCTGCGGCCTATTACATCATAGT | 156 |
| NEAT1_P5 | /5hexynyl/TCACTCCTCAGACCAACATCATAGT | 157 |
| NEAT1_P6 | /5hexynyl/AACACCCGAGCAGCGACATCATAGT | 158 |
| NEAT1_P7 | /5hexynyl/ACTCACACTGGCCAGACATCATAGT | 159 |
| NEAT1_P8 | /5hexynyl/GAATACAGGGCTTCCACATCATAGT | 160 |
| NEAT1_P9 | /5hexynyl/CTAACGAAGTAAAGTACATCATAGT | 161 |
| NEAT1_P10 | /5hexynyl/GATACTAGATAGGCAACATCATAGT | 162 |
| backbone_ | | |
| sequence | | |
| (P9 | | |
| backbone) | | |
| NEAT1_ | /5Phos/atattcgcagtgcattactggtAGTGGT | 101 |
| exon_ | AGGTAGACATTCatagtacagatgcattactggtA | |
| P1-P10 | GTGGTAGGTAGACATTCctgagtgttg | |
| rightarm_ | | |
| sequence | | |
| NEAT1_P1 | /5Phos/aagtgactgtTGTCCCCTGAAGCCC/ | 163 |
| | 3AzideN/ | |
| NEAT1_P2 | /5Phos/aagtgactgtCAAGAGCGGCGCCCT/ | 164 |
| | 3AzideN/ | |
| NEAT1_P3 | /5Phos/aagtgactgtAGACCAGCGCACCCG/ | 165 |
| | 3AzideN/ | |
| NEAT1_P4 | /5Phos/aagtgactgtTGATCATTTCCAGGG/ | 166 |
| | 3AzideN/ | |
| NEAT1_P5 | /5Phos/aagtgactgtCGCCTTAACTCCACA/ | 167 |
| | 3AzideN/ | |
| NEAT1_P6 | /5Phos/aagtgactgtGTCAATGTTGTCCCC/ | 168 |
| | 3AzideN/ | |
| NEAT1_P7 | /5Phos/aagtgactgtTCCTGCAATGCTAGG/ | 169 |
| | 3AzideN/ | |
| NEAT1_P8 | /5Phos/aagtgactgtAAATGGTAGCCTCCT/ | 170 |
| | 3AzideN/ | |
| NEAT1_P9 | /5Phos/aagtgactgtAAAGTATTATGAAAT/ | 171 |
| | 3AzideN/ | |
| NEAT1_P10 | /5Phos/aagtgactgtAGACTACAATTTGAA/ | 172 |
| | 3AzideN/ | |
| left_ | | |
| adapter | | |
| NEAT1_P1- | ctgcgaatatACTATGATGT | 112 |
| P10 | | |
| right_ | | |
| adapter | | |
| NEAT1_P1- | acagtcacttcaacactcag | 113 |
| P10 | | |

Nucleotides that are underlined represent regions which are complementary to the left or right adapters, in an embodiment. The italicized and bolded nucleotides are complementary to the left arm and right arm binding sequence respectively for subsequent amplification probe (e.g., secondary clampFISH probe, tertiary clampFISH probe), in an embodiment.

TABLE 8
Sequences of primary TMSF1 exon clampFISH probes.

| Primary | | |
|---|---|---|
| TMSF1 | | SEQ |
| clampFISH | | ID |
| probes: | | NO: |
| leftarm_ | | |
| sequence | | |
| TMSF1_exon_ | /5Hexynyl/aaagcaaacaccactACATCATAGT | 173 |
| P1 | | |
| TMSF1_exon_ | /5Hexynyl/aaaagaaccacacgaACATCATAGT | 174 |
| P2 | | |
| TMSF1_exon_ | /5Hexynyl/gtagccagatcctgcACATCATAGT | 175 |
| P3 | | |
| TMSF1_exon_ | /5Hexynyl/caattccaccaagagACATCATAGT | 176 |
| P4 | | |
| TMSF1_exon_ | /5Hexynyl/acatcctgtgaagatACATCATAGT | 177 |
| P5 | | |
| TMSF1_exon_ | /5Hexynyl/atatactgagtactgACATCATAGT | 178 |
| P6 | | |
| TMSF1_exon_ | /5Hexynyl/tacaagcagtgaaaaACATCATAGT | 179 |
| P7 | | |
| TMSF1_exon_ | /5Hexynyl/gattggaacattctcACATCATAGT | 180 |
| P8 | | |
| TMSF1_exon_ | /5Hexynyl/ttgagggtatttcctACATCATAGT | 181 |
| P9 | | |
| backbone_ | | |
| sequence | | |
| (P9 | | |
| backbone) | | |
| TMSF1_exon_ | /5Phos/atattcgcagtgcattactggtAGTGGT | 101 |
| P1-P9 | AGGTAGACATTCatagtacagatgcattactggtA | |
| | GTGGTAGGTAGACATTCctgagtgttg | |
| rightarm_ | | |
| sequence | | |
| TMSF1_exon_ | /5Phos/aagtgactgtgcccttctggtggag/ | 182 |
| P1 | 3AzideN/ | |
| TMSF1_exon_ | /5Phos/aagtgactgtctcctacgatgccag/ | 183 |
| P2 | 3AzideN/ | |
| TMSF1_exon_ | /5Phos/aagtgactgttgccacaatgacaca/ | 184 |
| P3 | 3AzideN/ | |
| TMSF1_exon_ | /5Phos/aagtgactgtgacacaagatgaatt/ | 185 |
| P4 | 3AzideN/ | |
| TMSF1_exon_ | /5Phos/aagtgactgttaaatttaaacactg/ | 186 |
| P5 | 3AzideN/ | |
| TMSF1_exon_ | /5Phos/aagtgactgttagagtttatctcag/ | 187 |
| P6 | 3AzideN/ | |
| TMSF1_exon_ | /5Phos/aagtgactgtgaatgggaaacatca/ | 188 |
| P7 | 3AzideN/ | |
| TMSF1_exon_ | /5Phos/aagtgactgttgatgcattcatttg/ | 189 |
| P8 | 3AzideN/ | |
| TMSF1_exon_ | /5Phos/aagtgactgtgtaaacaagttagtt/ | 190 |
| P9 | 3AzideN/ | |
| left_ | | |
| adapter | | |
| TMSF1_exon_ | ctgcgaatatACTATGATGT | 112 |
| P1-P9 | | |
| right_ | | |
| adapter | | |
| TMSF1_exon_ | acagtcacttcaacactcag | 113 |
| P1-P9 | | |

Nucleotides that are underlined represent regions which are complementary to the left or right adapters, in an embodiment. The italicized and bolded nucleotides are complementary to the left arm and right arm binding sequence respectively for subsequent amplification probe (e.g., secondary clampFISH probe, tertiary clampFISH probe), in an embodiment.

### Flow Cytometry and Sorting

For nuclampFISH, EEF2 intron and NEAT1 were sorted based on the nuclampFISH signal using the BECKMAN Sorter (Cell Sorter), which uses 638 nm excitation and 660 nm emission.

### DSP Cross-Linking Reversal

To reverse the DSP crosslinking for chromatin accessibility assays, the sorted cells were treated with 25 mM DTT at 37° C. for 30 minutes, according to the manufacturer's instructions. The cells were then washed with 1×PBS once and ready for the chromatin accessibility assay.

### RNA Extraction, RT-qPCR

RNA was extracted using Trizol (Thermo Fisher #15596026) and resuspended in NF water. DNase (Thermo Fisher #AM1907) was used to remove the contaminant DNA, RNA was reverse transcribed (Fisher Scientific #18-080-044). The Luna qPCR (NEB #M3004) was used to quantify the generated cDNA. The OneTaq RT-PCR mix (NEB #E5315) was used to amplify the generated cDNA. Sequences for primers can be found in Tables 9-10.

TABLE 9
EEF2 intron RT-qPCR primer and probes.

| EEF2 intron | | |
|---|---|---|
| RT-qPCR | | SEQ |
| primer and | | ID |
| probes | | NO: |
| Forward | tcagtcgggagctagaagtaa | 191 |
| primer | | |
| Reverse | cctcactgggctaagaacaaa | 192 |
| primer | | |
| Probe | /56-FAM/ATAGGGTGA/ZEN/ | 193 |
| | TACTTGTGCCAGCCA/3IABKFQ/ | |

“ZEN” refers to N,N-diethyl-4-(4-nitronaphthalen-1-ylazo)-phenylamine.
“56-FAM” refers to 6-carboxyfluorescein (6-FAM) attached at the 5′ end of the probe.
“3IABKFQ” refers to Iowa Black FQ quencher attached at the 3′ end of the probe.

TABLE 10
HPRT1 RT-qPCR primer and probes.

| HPRT1 | | |
|---|---|---|
| RT-qPCR | | |
| primer | | SEQ |
| and | | ID |
| probes | | NO: |
| Forward | cgagatgtgatgaaggagatgg | 194 |
| primer | | |
| Reverse | ttgatgtaatccagcaggtcag | 195 |
| primer | | |
| Probe | /56-FAM/ACAGAGGGC/ZEN/ | 196 |
| | TACAATGTGATGGCC/3IABKFQ/ | |

“ZEN” refers to N,N-diethyl-4-(4-nitronaphthalen-1-ylazo)-phenylamine.
“56-FAM” refers to 6-carboxyfluorescein (6-FAM) attached at the 5′ end of the probe.
“3IABKFQ” refers to Iowa Black FQ quencher attached at the 3′ end of the probe.

### Chromatin Accessibility Assay

After nuclei were sorted out based on the nuclampFISH signal and DSP crosslinking was cleaved, the cell's chromatin was extracted, and the chromatin accessibility assay (EpiQuik Chromatin Accessibility Assay Kit) was performed according to the manufacture protocol (Epigentek, #P-1047-48). The changes were made so that the chromatin with/without nuclease treatment was quantified by the previous qPCR protocol [28], and the housekeeping gene HPRT1 was set as a control.

### Image Acquisition

Microscopy was performed using a Nikon inverted research microscope eclipse Ti2-E/Ti2-E/B using a Plan Apo λ 20×/0.75 objective or Plan Apo λ 100×/1.45 oil objective. The Epi-fi LED illuminator linked to the microscope assured illumination and controlled the respective brightness of four types of LEDs of different wavelengths. Images were acquired using the Neutral Density (ND16) filter for probes coupled with Alexa 488, Alexa 594, Alexa 647, and cy3. Images were acquired and processed using ImageJ. Images acquired using the Neutral Density (ND16) filter are false-colored gray.

### Image Analysis and Quantification

Images were initially segmented and thresholded using a custom Matlab software suite (rajlabimage tools: https_github_com/arjunrajlaboratory/rajlabimagetools/wiki). Cell segmentation was performed manually by drawing boundaries around non-overlapping cells. The software then fits each detected spot to a two-dimensional Gaussian profile, specifically on the z plane on which it occurs, to ascertain subpixel-resolution spot locations. Colocalization took place in two stages. In the first stage, the software searched for the nearest neighbor for the smFISH probe with the nuclampFISH probe within a 2.5-pixel (360-nm) window to determine colocalization.

### REFERENCES

1. Raj, A., Peskin, C. S., Tranchina, D., Vargas, D. Y. & Tyagi, S. Stochastic mRNA synthesis in mammalian cells. PLoS Biol. 4, e309 (2006).
2. Zenklusen, D., Larson, D. R. & Singer, R. H. Single-RNA counting reveals alternative modes of gene expression in yeast. Nat. Struct. Mol. Biol. 15, 1263-1271 (2008).
3. Suter, D. M. et al. Mammalian genes are transcribed with widely different bursting kinetics. Science 332, 472-474 (2011).
4. Coleman, R. A. et al. Imaging Transcription: Past, Present, and Future. Cold Spring Harb. Symp. Quant. Biol. 80, 1-8 (2015).
5. Raj, A., Rifkin, S. A., Andersen, E. & van Oudenaarden, A. Variability in gene expression underlies incomplete penetrance. Nature 463, 913-918 (2010).
6. Park, P. J. ChIP-seq: advantages and challenges of a maturing technology. Nat. Rev. Genet. 10, 669-680 (2009).
7. Johnson, D. S., Mortazavi, A., Myers, R. M. & Wold, B. Genome-wide mapping of in vivo protein-DNA interactions. Science 316, 1497-1502 (2007).
8. Wissink, E. M., Vihervaara, A., Tippens, N. D. & Lis, J. T. Nascent RNA analyses: tracking transcription and its regulation. Nat. Rev. Genet. 20, 705-723 (2019).
9. Wheat, J. C. et al. Single-molecule imaging of transcription dynamics in somatic stem cells. Nature 583, 431-436 (2020).
10. Senecal, A. et al. Transcription factors modulate c-Fos transcriptional bursts. Cell Rep. 8, 75-83 (2014).
11. Choi, H. M. T. et al. Third-generation in situ hybridization chain reaction: multiplexed, quantitative, sensitive, versatile, robust. Development 145, (2018).
12. Garcia-Perez, L. et al. A Novel Branched DNA-Based Flowcytometric Method for Single-Cell Characterization of Gene Therapy Products and Expression of Therapeutic Genes. Front. Immunol. 11, 607991 (2020).
13. Kishi, J. Y. et al. SABER amplifies FISH: enhanced multiplexed imaging of RNA and DNA in cells and tissues. Nat. Methods 16, 533-544 (2019).
14. Rouhanifard, S. H. et al. ClampFISH detects individual nucleic acid molecules using click chemistry-based amplification. Nat. Biotechnol. 37, 84-89 (2018).
15. Dardani, I. et al. ClampFISH 2.0 enables rapid, scalable amplified RNA detection in situ. Nat. Methods 19, 1403-1410 (2022).
16. Post-transcriptional splicing can occur in a slow-moving zone around the gene. https://elifesciences.org/reviewed-preprints/91357.
17. Kotake, Y. et al. Splicing factor SF3b as a target of the antitumor natural product pladienolide. Nat. Chem. Biol. 3, 570-575 (2007).
18. Femino, A. M., Fay, F. S., Fogarty, K. & Singer, R. H. Visualization of single RNA transcripts in situ. Science 280, 585-590 (1998).
19. Raj, A., van den Bogaard, P., Rifkin, S. A., van Oudenaarden, A. & Tyagi, S. Imaging individual mRNA molecules using multiple singly labeled probes. Nat. Methods 5, 877-879 (2008).
20. Liu, C.-W. et al. Accurate Measurement of Formaldehyde-Induced DNA-Protein Cross-Links by High-Resolution Orbitrap Mass Spectrometry. Chem. Res. Toxicol. 31, 350-357 (2018).
21. Tayri-Wilk, T. et al. Mass spectrometry reveals the chemistry of formaldehyde cross-linking in structured proteins. Nat. Commun. 11, 3128 (2020).
22. Ongay, S. et al. Cleavable Crosslinkers as Tissue Fixation Reagents for Proteomic Analysis. Chembiochem 19, 736-743 (2018).
23. Tsompana, M. & Buck, M. J. Chromatin accessibility: a window into the genome. Epigenetics Chromatin 7, 33 (2014).
24. Mansisidor, A. R. & Risca, V. I. Chromatin accessibility: methods, mechanisms, and biological insights. Nucleus 13, 236-276 (2022).
25. Berrocal, A., Lammers, N. C., Garcia, H. G. & Eisen, M. B. Kinetic sculpting of the seven stripes of the Drosophila even-skipped gene. Elife 9, (2020).
26. Pandya-Jones, A. & Black, D. L. Co-transcriptional splicing of constitutive and alternative exons. RNA 15, 1896-1908 (2009).
27. Schwarzkopf, M. et al. Hybridization chain reaction enables a unified approach to multiplexed, quantitative, high-resolution immunohistochemistry and in situ hybridization. Development 148, (2021).
28. Liu, Y. et al. Paired Capture and FISH Detection of Individual Virions Enable Cell-Free Determination of Infectious Titers. ACS Sens 8, 2563-2571 (2023).
29. Liu, Y. et al. NuclampFISH enables cell sorting based on nuclear RNA expression for chromatin analysis. bioRxiv (2024).

The teachings of all patents, published applications and references cited herein or in the manuscript being filed herewith are incorporated by reference in their entirety.

While example embodiments have been particularly shown and described, it will be understood by those skilled in the art that various changes in form and details may be made therein without departing from the scope of the embodiments encompassed or contemplated herein or in the manuscript being filed herewith.

1. A method of labeling nuclear RNA, the method comprising:
(a) isolating a nucleus of a cell, the nucleus comprising nuclear RNA;
(b) contacting the nucleus with a fixative, thereby producing a fixed nucleus;
(c) contacting the fixed nucleus with a surfactant; and
(d) hybridizing a primary probe to the nuclear RNA, thereby forming a primary sample comprising labeled nuclear RNA.

2. The method of claim 1, wherein the primary probe is a primary click-amplified Fluorescence In Situ Hybridization (clampFISH) probe, a Hybridization Chain Reaction (HCR) probe, or a Systematic Amplification of Bioorthogonal Reporting (SABER) probe.

3. The method of claim 1, further comprising:
(e) contacting the primary sample with an agent that locks the primary probe to the nuclear RNA.

4. The method of claim 3, wherein the primary sample is contacted with the agent for at least about 10 minutes at about 37° C.

5. The method of claim 3, wherein the agent is a click chemistry agent catalyzing a cycloaddition between 5′ and 3′ end of the primary probe, thereby locking the primary probe to the nuclear RNA.

6. The method of claim 3, further comprising:
(f) contacting the primary sample with (i) a set of secondary probes or (ii) a set of secondary probes and an agent that locks the set of secondary probes to the primary probe, thereby forming a secondary sample.

7. The method of claim 6, wherein the secondary probes are secondary clampFISH probes, HCR probes, or SABER probes.

8. The method of claim 6, further comprising:
(g) contacting the secondary sample with (i) a set of tertiary probes or (ii) a set of tertiary probes and an agent that locks the set of tertiary probes to each secondary probe, thereby forming a tertiary sample;
(h) contacting the tertiary sample with (i) a set of secondary probes or (ii) a set of secondary probes and an agent that locks the set of secondary probes to each tertiary probe; and
(i) repeating steps (g) and (h) until a desired level of fluorescent signal is achieved.

9. The method of claim 8, wherein the tertiary probes are tertiary clampFISH probes, HCR probes, or SABER probes.

10. The method of claim 1, wherein the isolating comprises contacting the cell with a lysis buffer comprising a surfactant.

11. (canceled)

12. The method of claim 10, wherein the isolating further comprises applying a mechanical force to the cell.

13. The method of claim 10, wherein the cell is contacted with the lysis buffer at about 4° C. for about 10 minutes.

14. The method of claim 1, wherein the fixative comprises dithiobis(succinimidyl propionate) or disuccinimidyl sulfoxide.

15. (canceled)

16. (canceled)

17. The method of claim 1, wherein the fixed nucleus is contacted with the surfactant for from about 5 minutes to about 30 minutes.

18. The method of claim 1, wherein the surfactant is a non-ionic surfactant.

19. (canceled)

20. (canceled)

21. The method of claim 1, wherein the primary probe is a DNA probe labeled with a fluorophore.

22. (canceled)

23. A method of detecting nuclear RNA, the method comprising:
(a) isolating a nucleus of a cell, the nucleus comprising a nuclear RNA;
(b) contacting the nucleus with a fixative, thereby producing a fixed nucleus;
(c) contacting the fixed nucleus with a surfactant;
(d) hybridizing a primary probe to the nuclear RNA, thereby forming a primary sample comprising labeled nuclear RNA; and
(e) detecting the labeled nuclear RNA.

24. The method of claim 23, wherein the detecting comprises using flow cytometry, microscopy, or a combination thereof.

25. A method of analyzing nuclear RNA, the method comprising:
(a) isolating a nucleus of a cell, the nucleus comprising nuclear RNA;
(b) contacting the nucleus with a fixative, thereby producing a fixed nucleus;
(c) contacting the fixed nucleus with a surfactant;
(d) hybridizing a primary probe to the nuclear RNA, thereby forming a primary sample comprising labeled nuclear RNA;
(e) detecting the nuclear RNA;
(f) contacting the fixed nucleus with a reducing agent, thereby unfixing the nucleus; and
(g) analyzing the nuclear RNA.

26. The method of claim 25, wherein the analyzing comprises using mass spectrometry, a biochemical assay, chromatin profiling, RNA sequencing, metabolomics, or a combination thereof.


SEQUENCE LISTING (196 sequences)

SEQ ID NO: 1 - DNA, 21 bp, synthetic construct
  source 1..21: mol_type = other DNA
  modified_base 21: mod_base = m5c
cccaggtagaaccgaaagaan

SEQ ID NO: 2 - DNA, 21 bp, synthetic construct
  source 1..21: mol_type = other DNA
  modified_base 21: mod_base = m5c
gtatcagtgaagcgtgtctcn

SEQ ID NO: 3 - DNA, 21 bp, synthetic construct
  source 1..21: mol_type = other DNA
  modified_base 21: mod_base = m5c
gctcgtagaagagggagatgn

SEQ ID NO: 4 - DNA, 21 bp, synthetic construct
  source 1..21: mol_type = other DNA
  modified_base 21: mod_base = m5c
tccgaggagaagtcgacatgn

SEQ ID NO: 5 - DNA, 21 bp, synthetic construct
  source 1..21: mol_type = other DNA
  modified_base 21: mod_base = m5c
cttgttcatcatcagcacagn

SEQ ID NO: 6 - DNA, 21 bp, synthetic construct
  source 1..21: mol_type = other DNA
  modified_base 21: mod_base = m5c
acgatgcgctggaaagtctgn

SEQ ID NO: 7 - DNA, 21 bp, synthetic construct
  source 1..21: mol_type = other DNA
  modified_base 21: mod_base = m5c
cgaggacaggatcgatcatgn

SEQ ID NO: 8 - DNA, 21 bp, synthetic construct
  source 1..21: mol_type = other DNA
  modified_base 21: mod_base = m5c
ttcatcatgtcctctactttn

SEQ ID NO: 9 - DNA, 21 bp, synthetic construct
  source 1..21: mol_type = other DNA
  modified_base 21: mod_base = m5c
tggctgacttgctgaacttgn

SEQ ID NO: 10 - DNA, 21 bp, synthetic construct
  source 1..21: mol_type = other DNA
  modified_base 21: mod_base = m5c
tgatgtccagtttctctatcn

SEQ ID NO: 11 - DNA, 21 bp, synthetic construct
  source 1..21: mol_type = other DNA
  modified_base 21: mod_base = m5c
tttggggtcacagcttttaan

SEQ ID NO: 12 - DNA, 21 bp, synthetic construct
  source 1..21: mol_type = other DNA
  modified_base 21: mod_base = m5c
gcccatcatcaagattgttcn

SEQ ID NO: 13 - DNA, 21 bp, synthetic construct
  source 1..21: mol_type = other DNA
  modified_base 21: mod_base = m5c
aatgttcccacaaggcacatn

SEQ ID NO: 14 - DNA, 21 bp, synthetic construct
  source 1..21: mol_type = other DNA
  modified_base 21: mod_base = m5c
cgtgctcgaaggtggtgatgn

SEQ ID NO: 15 - DNA, 21 bp, synthetic construct
  source 1..21: mol_type = other DNA
  modified_base 21: mod_base = m5c
actctgacaacagggctgacn

SEQ ID NO: 16 - DNA, 21 bp, synthetic construct
  source 1..21: mol_type = other DNA
  modified_base 21: mod_base = m5c
cgatgatgcactgcaccatgn

SEQ ID NO: 17 - DNA, 21 bp, synthetic construct
  source 1..21: mol_type = other DNA
  modified_base 21: mod_base = m5c
gagacgaccgggtcagatttn

SEQ ID NO: 18 - DNA, 21 bp, synthetic construct
  source 1..21: mol_type = other DNA
  modified_base 21: mod_base = m5c
ttgttgggggacttggagagn

SEQ ID NO: 19 - DNA, 21 bp, synthetic construct
  source 1..21: mol_type = other DNA
  modified_base 21: mod_base = m5c
caaagcaccagatcttgcggn

SEQ ID NO: 20 - DNA, 21 bp, synthetic construct
  source 1..21: mol_type = other DNA
  modified_base 21: mod_base = m5c
tgatgtcggtgaggatgttgn

SEQ ID NO: 21 - DNA, 21 bp, synthetic construct
  source 1..21: mol_type = other DNA
  modified_base 21: mod_base = m5c
cttgatctcgttgaggtactn

SEQ ID NO: 22 - DNA, 21 bp, synthetic construct
  source 1..21: mol_type = other DNA
  modified_base 21: mod_base = m5c
tgacgtcgtggacgtcgaagn

SEQ ID NO: 23 - DNA, 21 bp, synthetic construct
  source 1..21: mol_type = other DNA
  modified_base 21: mod_base = m5c
gtcagcacactggcatagagn

SEQ ID NO: 24 - DNA, 21 bp, synthetic construct
  source 1..21: mol_type = other DNA
  modified_base 21: mod_base = m5c
aaggtagatgggctccatgan

SEQ ID NO: 25 - DNA, 21 bp, synthetic construct
  source 1..21: mol_type = other DNA
  modified_base 21: mod_base = m5c
tgttcaaaaccccgtagatgn

SEQ ID NO: 26 - DNA, 21 bp, synthetic construct
  source 1..21: mol_type = other DNA
  modified_base 21: mod_base = m5c
cctacaatttgtccaggaagn

SEQ ID NO: 27 - DNA, 21 bp, synthetic construct
  source 1..21: mol_type = other DNA
  modified_base 21: mod_base = m5c
tccaggtgtcgtctgagaatn

SEQ ID NO: 28 - DNA, 21 bp, synthetic construct
  source 1..21: mol_type = other DNA
  modified_base 21: mod_base = m5c
ccattaagtccctactaagan

SEQ ID NO: 29 - DNA, 21 bp, synthetic construct
  source 1..21: mol_type = other DNA
  modified_base 21: mod_base = m5c
ttctggggcaaaagccactgn

SEQ ID NO: 30 - DNA, 21 bp, synthetic construct
  source 1..21: mol_type = other DNA
  modified_base 21: mod_base = m5c
caatggcactagtacagctgn

SEQ ID NO: 31 - DNA, 21 bp, synthetic construct
  source 1..21: mol_type = other DNA
  modified_base 21: mod_base = m5c
cctcggaaacggagaaagggn

SEQ ID NO: 32 - DNA, 21 bp, synthetic construct
  source 1..21: mol_type = other DNA
  modified_base 21: mod_base = m5c
ttggcaaacgggatcagacan

SEQ ID NO: 33 - DNA, 21 bp, synthetic construct
  source 1..21: mol_type = other DNA
  modified_base 21: mod_base = m5c
atttggggccaaacacgtaan

SEQ ID NO: 34 - DNA, 21 bp, synthetic construct
  source 1..21: mol_type = other DNA
  modified_base 21: mod_base = m5c
ttccttgataccaggtacaan

SEQ ID NO: 35 - DNA, 21 bp, synthetic construct
  source 1..21: mol_type = other DNA
  modified_base 21: mod_base = m5c
ccaaggaaccaccgttaatan

SEQ ID NO: 36 - DNA, 21 bp, synthetic construct
  source 1..21: mol_type = other DNA
  modified_base 21: mod_base = m5c
agcgttcagtccaaacgaacn

SEQ ID NO: 37 - DNA, 21 bp, synthetic construct
  source 1..21: mol_type = other DNA
  modified_base 21: mod_base = m5c
tcgtgatttccaggaacacan

SEQ ID NO: 38 - DNA, 21 bp, synthetic construct
  source 1..21: mol_type = other DNA
  modified_base 21: mod_base = m5c
ctcaagaaaaccttccagctn

SEQ ID NO: 39 - DNA, 21 bp, synthetic construct
  source 1..21: mol_type = other DNA
  modified_base 21: mod_base = m5c
agatattgtaggagtgggggn

SEQ ID NO: 40 - DNA, 21 bp, synthetic construct
  source 1..21: mol_type = other DNA
  modified_base 21: mod_base = m5c
gaacatcatccagtgcaacan

SEQ ID NO: 41 - DNA, 21 bp, synthetic construct
  source 1..21: mol_type = other DNA
  modified_base 21: mod_base = m5c
ctaaaagacccagcaggacgn

SEQ ID NO: 42 - DNA, 21 bp, synthetic construct
  source 1..21: mol_type = other DNA
  modified_base 21: mod_base = m5c
agattcatcacacctgactcn

SEQ ID NO: 43 - DNA, 21 bp, synthetic construct
  source 1..21: mol_type = other DNA
  modified_base 21: mod_base = m5c
aaaaacacacttccagtcccn

SEQ ID NO: 44 - DNA, 21 bp, synthetic construct
  source 1..21: mol_type = other DNA
  modified_base 21: mod_base = m5c
tcttcagtagacatctggtcn

SEQ ID NO: 45 - DNA, 21 bp, synthetic construct
  source 1..21: mol_type = other DNA
  modified_base 21: mod_base = m5c
aaacgagaagcagccgtgagn

SEQ ID NO: 46 - DNA, 21 bp, synthetic construct
  source 1..21: mol_type = other DNA
  modified_base 21: mod_base = m5c
gcagtccagatcttaagagan

SEQ ID NO: 47 - DNA, 21 bp, synthetic construct
  source 1..21: mol_type = other DNA
  modified_base 21: mod_base = m5c
gccaaagccacaagttattcn

SEQ ID NO: 48 - DNA, 21 bp, synthetic construct
  source 1..21: mol_type = other DNA
  modified_base 21: mod_base = m5c
gaagccaaggaagccaaagtn

SEQ ID NO: 49 - DNA, 21 bp, synthetic construct
  source 1..21: mol_type = other DNA
  modified_base 21: mod_base = m5c
gacagagaaaacccgcaagcn

SEQ ID NO: 50 - DNA, 21 bp, synthetic construct
  source 1..21: mol_type = other DNA
  modified_base 21: mod_base = m5c
tctatgctccttacttctagn

SEQ ID NO: 51 - DNA, 21 bp, synthetic construct
  source 1..21: mol_type = other DNA
  modified_base 21: mod_base = m5c
acaaaagttgtggctggcacn

SEQ ID NO: 52 - DNA, 21 bp, synthetic construct
  source 1..21: mol_type = other DNA
  modified_base 21: mod_base = m5c
ccacctggaagaagcagaagn

SEQ ID NO: 53 - DNA, 21 bp, synthetic construct
  source 1..21: mol_type = other DNA
  modified_base 21: mod_base = m5c
gccagaacaagatttcagggn

SEQ ID NO: 54 - DNA, 21 bp, synthetic construct
  source 1..21: mol_type = other DNA
  modified_base 21: mod_base = m5c
tttaaagcagagggcaggtgn

SEQ ID NO: 55 - DNA, 21 bp, synthetic construct
  source 1..21: mol_type = other DNA
  modified_base 21: mod_base = m5c
agcatttgtgttgtttgtggn

SEQ ID NO: 56 - DNA, 21 bp, synthetic construct
  source 1..21: mol_type = other DNA
  modified_base 21: mod_base = m5c
ctcactccagacaaggacaan

SEQ ID NO: 57 - DNA, 21 bp, synthetic construct
  source 1..21: mol_type = other DNA
  modified_base 21: mod_base = m5c
gccacgtcaatcagaacgaan

SEQ ID NO: 58 - DNA, 21 bp, synthetic construct
  source 1..21: mol_type = other DNA
  modified_base 21: mod_base = m5c
tgcctcacctgcaaaaagagn

SEQ ID NO: 59 - DNA, 21 bp, synthetic construct
  source 1..21: mol_type = other DNA
  modified_base 21: mod_base = m5c
acctgccagaagggaaagaan

SEQ ID NO: 60 - DNA, 21 bp, synthetic construct
  source 1..21: mol_type = other DNA
  modified_base 21: mod_base = m5c
attcagtgatttagggggtcn

SEQ ID NO: 61 - DNA, 21 bp, synthetic construct
  source 1..21: mol_type = other DNA
  modified_base 21: mod_base = m5c
tcctacctatagagtttagcn

SEQ ID NO: 62 - DNA, 21 bp, synthetic construct
  source 1..21: mol_type = other DNA
  modified_base 21: mod_base = m5c
actattagcttcagacaccgn

SEQ ID NO: 63 - DNA, 21 bp, synthetic construct
  source 1..21: mol_type = other DNA
  modified_base 21: mod_base = m5c
ctgtggttgaagctaatgctn

SEQ ID NO: 64 - DNA, 21 bp, synthetic construct
  source 1..21: mol_type = other DNA
  modified_base 21: mod_base = m5c
agatttgccatgccataatcn

SEQ ID NO: 65 - DNA, 21 bp, synthetic construct
  source 1..21: mol_type = other DNA
  modified_base 21: mod_base = m5c
gaccccagataacaaactctn

SEQ ID NO: 66 - DNA, 21 bp, synthetic construct
  source 1..21: mol_type = other DNA
  modified_base 21: mod_base = m5c
cacagctacatcttaggagtn

SEQ ID NO: 67 - DNA, 21 bp, synthetic construct
  source 1..21: mol_type = other DNA
  modified_base 21: mod_base = m5c
tttgatagagctgccactatn

SEQ ID NO: 68 - DNA, 21 bp, synthetic construct
  source 1..21: mol_type = other DNA
  modified_base 21: mod_base = m5c
tgcctgagtcagtgagaatan

SEQ ID NO: 69 - DNA, 21 bp, synthetic construct
  source 1..21: mol_type = other DNA
  modified_base 21: mod_base = m5c
tcaggcaattgttctggtatn

SEQ ID NO: 70 - DNA, 21 bp, synthetic construct
  source 1..21: mol_type = other DNA
  modified_base 21: mod_base = m5c
attcatcaccacatacatccn

SEQ ID NO: 71 - DNA, 21 bp, synthetic construct
  source 1..21: mol_type = other DNA
  modified_base 21: mod_base = m5c
gtacatccagtatgttctgan

SEQ ID NO: 72 - DNA, 21 bp, synthetic construct
  source 1..21: mol_type = other DNA
  modified_base 21: mod_base = m5c
tgcattctggttgcataagtn

SEQ ID NO: 73 - DNA, 21 bp, synthetic construct
  source 1..21: mol_type = other DNA
  modified_base 21: mod_base = m5c
cccatattgaaatccttggan

SEQ ID NO: 74 - DNA, 21 bp, synthetic construct
  source 1..21: mol_type = other DNA
  modified_base 21: mod_base = m5c
gtggggttttaatcttagggn

SEQ ID NO: 75 - DNA, 21 bp, synthetic construct
  source 1..21: mol_type = other DNA
  modified_base 21: mod_base = m5c
acttccttccacaacagaacn

SEQ ID NO: 76 - DNA, 21 bp, synthetic construct
  source 1..21: mol_type = other DNA
  modified_base 21: mod_base = m5c
tcagcaaaccatactgaggtn

SEQ ID NO: 77 - DNA, 21 bp, synthetic construct
  source 1..21: mol_type = other DNA
  modified_base 21: mod_base = m5c
acacacggtcatacttgaggn

SEQ ID NO: 78 - DNA, 21 bp, synthetic construct
  source 1..21: mol_type = other DNA
  modified_base 21: mod_base = m5c
tggtatattaacaccagggcn

SEQ ID NO: 79 - DNA, 21 bp, synthetic construct
  source 1..21: mol_type = other DNA
  modified_base 21: mod_base = m5c
gaagcttgttactgacagctn

SEQ ID NO: 80 - DNA, 21 bp, synthetic construct
  source 1..21: mol_type = other DNA
  modified_base 21: mod_base = m5c
cagcatgttcttgtgtacatn

SEQ ID NO: 81 - DNA, 21 bp, synthetic construct
  source 1..21: mol_type = other DNA
  modified_base 21: mod_base = m5c
ttttgcacagagcgactcaan

SEQ ID NO: 82 - DNA, 21 bp, synthetic construct
  source 1..21: mol_type = other DNA
  modified_base 21: mod_base = m5c
ttcctggttttctttcacaan

SEQ ID NO: 83 - DNA, 21 bp, synthetic construct
  source 1..21: mol_type = other DNA
  modified_base 21: mod_base = m5c
ccctctcaaagataggatcan

SEQ ID NO: 84 - DNA, 21 bp, synthetic construct
  source 1..21: mol_type = other DNA
  modified_base 21: mod_base = m5c
gctatacttgcctgataaggn

SEQ ID NO: 85 - DNA, 21 bp, synthetic construct
  source 1..21: mol_type = other DNA
  modified_base 21: mod_base = m5c
cacttactttgaactctgccn

SEQ ID NO: 86 - DNA, 21 bp, synthetic construct
  source 1..21: mol_type = other DNA
  modified_base 21: mod_base = m5c
tttggtcattggctctttatn

SEQ ID NO: 87 - DNA, 21 bp, synthetic construct
  source 1..21: mol_type = other DNA
  modified_base 21: mod_base = m5c
ctgggaaacctggtgtatttn

SEQ ID NO: 88 - DNA, 21 bp, synthetic construct
  source 1..21: mol_type = other DNA
  modified_base 21: mod_base = m5c
gcaggatactgatattgggtn

SEQ ID NO: 89 - DNA, 21 bp, synthetic construct
  source 1..21: mol_type = other DNA
  modified_base 21: mod_base = m5c
aaacaaacccagatcccttgn

SEQ ID NO: 90 - DNA, 21 bp, synthetic construct
  source 1..21: mol_type = other DNA
  modified_base 21: mod_base = m5c
tcttagcttcagcaaagcttn

SEQ ID NO: 91 - DNA, 25 bp, synthetic construct
  source 1..25: mol_type = other DNA
tgttgcggatgttggacatcatagt

SEQ ID NO: 92 - DNA, 25 bp, synthetic construct
  source 1..25: mol_type = other DNA
tgatgaggaagccggacatcatagt

SEQ ID NO: 93 - DNA, 25 bp, synthetic construct
  source 1..25: mol_type = other DNA
gatgacgttcacgttacatcatagt

SEQ ID NO: 94 - DNA, 25 bp, synthetic construct
  source 1..25: mol_type = other DNA
caaactgcttcagggacatcatagt

SEQ ID NO: 95 - DNA, 25 bp, synthetic construct
  source 1..25: mol_type = other DNA
ttgaagatggggtccacatcatagt

SEQ ID NO: 96 - DNA, 25 bp, synthetic construct
  source 1..25: mol_type = other DNA
tctgcaacaaggcgtacatcatagt

SEQ ID NO: 97 - DNA, 25 bp, synthetic construct
  source 1..25: mol_type = other DNA
accgacctttgtcggacatcatagt

SEQ ID NO: 98 - DNA, 25 bp, synthetic construct
  source 1..25: mol_type = other DNA
gactcgttgacgggcacatcatagt

SEQ ID NO: 99 - DNA, 25 bp, synthetic construct
  source 1..25: mol_type = other DNA
gttgagtgatggcacacatcatagt

SEQ ID NO: 100 - DNA, 25 bp, synthetic construct
  source 1..25: mol_type = other DNA
aaatcatctacccgcacatcatagt

SEQ ID NO: 101 - DNA, 90 bp, synthetic construct
  source 1..90: mol_type = other DNA
atattcgcagtgcattactggtagtggtaggtagacattcatagtacagatgcattactggtagtggtaggtagacattcctgagtgttg

SEQ ID NO: 102 - DNA, 25 bp, synthetic construct
  source 1..25: mol_type = other DNA
aagtgactgtgggcgatgacagaca

SEQ ID NO: 103 - DNA, 25 bp, synthetic construct
  source 1..25: mol_type = other DNA
aagtgactgtgggagtcaatgaggt

SEQ ID NO: 104 - DNA, 25 bp, synthetic construct
  source 1..25: mol_type = other DNA
aagtgactgtgccgtaggtggagat

SEQ ID NO: 105 - DNA, 25 bp, synthetic construct
  source 1..25: mol_type = other DNA
aagtgactgtccacatacatctcgg

SEQ ID NO: 106 - DNA, 25 bp, synthetic construct
  source 1..25: mol_type = other DNA
aagtgactgtatcgcatcaaacacc

SEQ ID NO: 107 - DNA, 25 bp, synthetic construct
  source 1..25: mol_type = other DNA
aagtgactgtggtggatggtgatca

SEQ ID NO: 108 - DNA, 25 bp, synthetic construct
  source 1..25: mol_type = other DNA
aagtgactgtgtccaaaggcgtaga

SEQ ID NO: 109 - DNA, 25 bp, synthetic construct
  source 1..25: mol_type = other DNA
aagtgactgtgcggtgaagccaaag

SEQ ID NO: 110 - DNA, 25 bp, synthetic construct
  source 1..25: mol_type = other DNA
aagtgactgtgcatcaagtgttatg

SEQ ID NO: 111 - DNA, 25 bp, synthetic construct
  source 1..25: mol_type = other DNA
aagtgactgtcttcctgcctgctag

SEQ ID NO: 112 - DNA, 20 bp, synthetic construct
  source 1..20: mol_type = other DNA
ctgcgaatatactatgatgt

SEQ ID NO: 113 - DNA, 20 bp, synthetic construct
  source 1..20: mol_type = other DNA
acagtcacttcaacactcag

SEQ ID NO: 114 - DNA, 25 bp, synthetic construct
  source 1..25: mol_type = other DNA
actaccagtaatgcattttaatatt

SEQ ID NO: 115 - DNA, 92 bp, synthetic construct
  source 1..92: mol_type = other DNA
caatgagttacaggagttgtgtttgtggacgaagagcaccatcatattaaggcaggagttgtgtttgtggacgaagagcaccataagaatca

SEQ ID NO: 116 - DNA, 25 bp, synthetic construct
  source 1..25: mol_type = other DNA
aagtgactgtgaatgtctacctacc

SEQ ID NO: 117 - DNA, 20 bp, synthetic construct
  source 1..20: mol_type = other DNA
taactcattgaatattaaaa

SEQ ID NO: 118 - DNA, 20 bp, synthetic construct
  source 1..20: mol_type = other DNA
acagtcactttgattcttat

SEQ ID NO: 119 - DNA, 25 bp, synthetic construct
  source 1..25: mol_type = other DNA
caaacacaactcctgacatcatagt

SEQ ID NO: 120 - DNA, 25 bp, synthetic construct
  source 1..25: mol_type = other DNA
aagtgactgtggtgctcttcgtcca

SEQ ID NO: 121 - DNA, 25 bp, synthetic construct
  source 1..25: mol_type = other DNA
gccgcgaccagacatacatcatagt

SEQ ID NO: 122 - DNA, 25 bp, synthetic construct
  source 1..25: mol_type = other DNA
cgcctcctttacccaacatcatagt

SEQ ID NO: 123 - DNA, 25 bp, synthetic construct
  source 1..25: mol_type = other DNA
ttatctcgcaggaccacatcatagt

SEQ ID NO: 124 - DNA, 25 bp, synthetic construct
  source 1..25: mol_type = other DNA
ccggatggcaggcttacatcatagt

SEQ ID NO: 125 - DNA, 25 bp, synthetic construct
  source 1..25: mol_type = other DNA
tgcgcttgacaccccacatcatagt

SEQ ID NO: 126 - DNA, 25 bp, synthetic construct
  source 1..25: mol_type = other DNA
ccgcgagtctcctcgacatcatagt

SEQ ID NO: 127 - DNA, 25 bp, synthetic construct
  source 1..25: mol_type = other DNA
aagtgactgttccctttccgccttt

SEQ ID NO: 128 - DNA, 25 bp, synthetic construct
  source 1..25: mol_type = other DNA
aagtgactgttacggtgacgcttag

SEQ ID NO: 129 - DNA, 25 bp, synthetic construct
  source 1..25: mol_type = other DNA
aagtgactgtgtaatgccctggatg

SEQ ID NO: 130 - DNA, 25 bp, synthetic construct
  source 1..25: mol_type = other DNA
aagtgactgtgcgacgagcaaggcg

SEQ ID NO: 131 - DNA, 25 bp, synthetic construct
  source 1..25: mol_type = other DNA
aagtgactgtagatgagaccagaaa

SEQ ID NO: 132 - DNA, 25 bp, synthetic construct
  source 1..25: mol_type = other DNA
aagtgactgtaacaccttcagaacc

SEQ ID NO: 133 - DNA, 25 bp, synthetic construct
  source 1..25: mol_type = other DNA
tctcaataagagtctacatcatagt

SEQ ID NO: 134 - DNA, 25 bp, synthetic construct
  source 1..25: mol_type = other DNA
cttcatcataaaatgacatcatagt

SEQ ID NO: 135 - DNA, 25 bp, synthetic construct
  source 1..25: mol_type = other DNA
taagaaaccaggggaacatcatagt

SEQ ID NO: 136 - DNA, 25 bp, synthetic construct
  source 1..25: mol_type = other DNA
gatgaaaacattccaacatcatagt

SEQ ID NO: 137 - DNA, 25 bp, synthetic construct
  source 1..25: mol_type = other DNA
aagttagggtctccaacatcatagt

SEQ ID NO: 138 - DNA, 25 bp, synthetic construct
  source 1..25: mol_type = other DNA
agaggaaaaagcccaacatcatagt

SEQ ID NO: 139 - DNA, 25 bp, synthetic construct
  source 1..25: mol_type = other DNA
atgttaagtcccacaacatcatagt

SEQ ID NO: 140 - DNA, 25 bp, synthetic construct
  source 1..25: mol_type = other DNA
taagaagctgagaccacatcatagt

SEQ ID NO: 141 - DNA, 25 bp, synthetic construct
  source 1..25: mol_type = other DNA
aacgaggtctggagcacatcatagt

SEQ ID NO: 142 - DNA, 25 bp, synthetic construct
  source 1..25: mol_type = other DNA
tcctagcaaggcggaacatcatagt

SEQ ID NO: 143 - DNA, 25 bp, synthetic construct
  source 1..25: mol_type = other DNA
aagtgactgtagcctacacctcgtg

SEQ ID NO: 144 - DNA, 25 bp, synthetic construct
  source 1..25: mol_type = other DNA
aagtgactgttccccgagcaagaag

SEQ ID NO: 145 - DNA, 25 bp, synthetic construct
  source 1..25: mol_type = other DNA
aagtgactgtcattctcccatgaat

SEQ ID NO: 146 - DNA, 25 bp, synthetic construct
  source 1..25: mol_type = other DNA
aagtgactgtggctgggtcaagtcg

SEQ ID NO: 147 - DNA, 25 bp, synthetic construct
  source 1..25: mol_type = other DNA
aagtgactgtaggccaagccaaatc

SEQ ID NO: 148 - DNA, 25 bp, synthetic construct
  source 1..25: mol_type = other DNA
aagtgactgtacctcgtggtggagc

SEQ ID NO: 149 - DNA, 25 bp, synthetic construct
  source 1..25: mol_type = other DNA
aagtgactgtcaaactcctgaagaa

SEQ ID NO: 150 - DNA, 25 bp, synthetic construct
  source 1..25: mol_type = other DNA
aagtgactgtgaaacttgaggaact

SEQ ID NO: 151 - DNA, 25 bp, synthetic construct
  source 1..25: mol_type = other DNA
aagtgactgtcattaacagggaaga

SEQ ID NO: 152 - DNA, 25 bp, synthetic construct
  source 1..25: mol_type = other DNA
aagtgactgtatccttaaaagcttt

SEQ ID NO: 153 - DNA, 25 bp, synthetic construct
  source 1..25: mol_type = other DNA
tgagctagccacttcacatcatagt

SEQ ID NO: 154 - DNA, 25 bp, synthetic construct
  source 1..25: mol_type = other DNA
cccagaggtcaagttacatcatagt

SEQ ID NO: 155 - DNA, 25 bp, synthetic construct
  source 1..25: mol_type = other DNA
ggctcgctcagctatacatcatagt

SEQ ID NO: 156 - DNA, 25 bp, synthetic construct
  source 1..25: mol_type = other DNA
ctgctgcggcctattacatcatagt

SEQ ID NO: 157 - DNA, 25 bp, synthetic construct
  source 1..25: mol_type = other DNA
tcactcctcagaccaacatcatagt

SEQ ID NO: 158 - DNA, 25 bp, synthetic construct
  source 1..25: mol_type = other DNA
aacacccgagcagcgacatcatagt

SEQ ID NO: 159 - DNA, 25 bp, synthetic construct
  source 1..25: mol_type = other DNA
actcacactggccagacatcatagt

SEQ ID NO: 160 - DNA, 25 bp, synthetic construct
  source 1..25: mol_type = other DNA
gaatacagggcttccacatcatagt

SEQ ID NO: 161 - DNA, 25 bp, synthetic construct
  source 1..25: mol_type = other DNA
ctaacgaagtaaagtacatcatagt

SEQ ID NO: 162 - DNA, 25 bp, synthetic construct
  source 1..25: mol_type = other DNA
gatactagataggcaacatcatagt

SEQ ID NO: 163 - DNA, 25 bp, synthetic construct
  source 1..25: mol_type = other DNA
aagtgactgttgtcccctgaagccc

SEQ ID NO: 164 - DNA, 25 bp, synthetic construct
  source 1..25: mol_type = other DNA
aagtgactgtcaagagcggcgccct

SEQ ID NO: 165 - DNA, 25 bp, synthetic construct
  source 1..25: mol_type = other DNA
aagtgactgtagaccagcgcacccg

SEQ ID NO: 166 - DNA, 25 bp, synthetic construct
  source 1..25: mol_type = other DNA
aagtgactgttgatcatttccaggg

SEQ ID NO: 167 - DNA, 25 bp, synthetic construct
  source 1..25: mol_type = other DNA
aagtgactgtcgccttaactccaca

SEQ ID NO: 168 - DNA, 25 bp, synthetic construct
  source 1..25: mol_type = other DNA
aagtgactgtgtcaatgttgtcccc

SEQ ID NO: 169 - DNA, 25 bp, synthetic construct
  source 1..25: mol_type = other DNA
aagtgactgttcctgcaatgctagg

SEQ ID NO: 170 - DNA, 25 bp, synthetic construct
  source 1..25: mol_type = other DNA
aagtgactgtaaatggtagcctcct

SEQ ID NO: 171 - DNA, 25 bp, synthetic construct
  source 1..25: mol_type = other DNA
aagtgactgtaaagtattatgaaat

SEQ ID NO: 172 - DNA, 25 bp, synthetic construct
  source 1..25: mol_type = other DNA
aagtgactgtagactacaatttgaa

SEQ ID NO: 173 - DNA, 25 bp, synthetic construct
  source 1..25: mol_type = other DNA
aaagcaaacaccactacatcatagt

SEQ ID NO: 174 - DNA, 25 bp, synthetic construct
  source 1..25: mol_type = other DNA
aaaagaaccacacgaacatcatagt

SEQ ID NO: 175 - DNA, 25 bp, synthetic construct
  source 1..25: mol_type = other DNA
gtagccagatcctgcacatcatagt

SEQ ID NO: 176 - DNA, 25 bp, synthetic construct
  source 1..25: mol_type = other DNA
caattccaccaagagacatcatagt

SEQ ID NO: 177 - DNA, 25 bp, synthetic construct
  source 1..25: mol_type = other DNA
acatcctgtgaagatacatcatagt

SEQ ID NO: 178 - DNA, 25 bp, synthetic construct
  source 1..25: mol_type = other DNA
atatactgagtactgacatcatagt

SEQ ID NO: 179 - DNA, 25 bp, synthetic construct
  source 1..25: mol_type = other DNA
tacaagcagtgaaaaacatcatagt

SEQ ID NO: 180 - DNA, 25 bp, synthetic construct
  source 1..25: mol_type = other DNA
gattggaacattctcacatcatagt

SEQ ID NO: 181 - DNA, 25 bp, synthetic construct
  source 1..25: mol_type = other DNA
ttgagggtatttcctacatcatagt

SEQ ID NO: 182 - DNA, 25 bp, synthetic construct
  source 1..25: mol_type = other DNA
aagtgactgtgcccttctggtggag

SEQ ID NO: 183 - DNA, 25 bp, synthetic construct
  source 1..25: mol_type = other DNA
aagtgactgtctcctacgatgccag

SEQ ID NO: 184 - DNA, 25 bp, synthetic construct
  source 1..25: mol_type = other DNA
aagtgactgttgccacaatgacaca

SEQ ID NO: 185 - DNA, 25 bp, synthetic construct
  source 1..25: mol_type = other DNA
aagtgactgtgacacaagatgaatt

SEQ ID NO: 186 - DNA, 25 bp, synthetic construct
  source 1..25: mol_type = other DNA
aagtgactgttaaatttaaacactg

SEQ ID NO: 187 - DNA, 25 bp, synthetic construct
  source 1..25: mol_type = other DNA
aagtgactgttagagtttatctcag

SEQ ID NO: 188 - DNA, 25 bp, synthetic construct
  source 1..25: mol_type = other DNA
aagtgactgtgaatgggaaacatca

SEQ ID NO: 189 - DNA, 25 bp, synthetic construct
  source 1..25: mol_type = other DNA
aagtgactgttgatgcattcatttg

SEQ ID NO: 190 - DNA, 25 bp, synthetic construct
  source 1..25: mol_type = other DNA
aagtgactgtgtaaacaagttagtt

SEQ ID NO: 191 - DNA, 21 bp, synthetic construct
  source 1..21: mol_type = other DNA
tcagtcgggagctagaagtaa

SEQ ID NO: 192 - DNA, 21 bp, synthetic construct
  source 1..21: mol_type = other DNA
cctcactgggctaagaacaaa

SEQ ID NO: 193 - DNA, 25 bp, synthetic construct
  source 1..25: mol_type = other DNA
  modified_base 10: mod_base = OTHER; note = ZEN modifier, N,N-diethyl-4-(4-nitronaphthalen-1-ylazo)-phenylamine
atagggtgantacttgtgccagcca

SEQ ID NO: 194 - DNA, 22 bp, synthetic construct
  source 1..22: mol_type = other DNA
cgagatgtgatgaaggagatgg

SEQ ID NO: 195 - DNA, 22 bp, synthetic construct
  source 1..22: mol_type = other DNA
ttgatgtaatccagcaggtcag

SEQ ID NO: 196 - DNA, 25 bp, synthetic construct
  source 1..25: mol_type = other DNA
  modified_base 10: mod_base = OTHER; note = ZEN modifier, N,N-diethyl-4-(4-nitronaphthalen-1-ylazo)-phenylamine
acagagggcntacaatgtgatggcc